(19) United States
(12) Patent Application Publication
(10) Pub. No.: US 2025/0369956 A1
(43) Pub. Date: Dec. 4, 2025

(54) THERAPY ASSESSMENT FOR HEMATOPOIETIC CANCER

(71) Applicants: Deutsches Krebsforschungszentrum Stiftung des öffentlichen Rechts, Heidelberg (DE); HI-STEM GGMBH, Heidelberg (DE)

(72) Inventors: Andreas TRUMPP, Heidelberg (DE); Alexander WACLAWICZEK, Heidelberg (DE); Aino-Maija LEPPÄ, Heidelberg (DE); Simon RENDERS, Heidelberg (DE); Carsten MÜLLER-TIDOW, Heidelberg (DE)

(73) Assignees: Deutsches Krebsforschungszentrum Stiftung des öffentlichen Rechts, Heidelberg (DE); HI-STEM GGMBH, Heidelberg (DE)

(21) Appl. No.: 18/872,773

(22) PCT Filed: Feb. 24, 2023
(86) PCT No.: PCT/EP2023/054685

§ 371 Date: Dec. 6, 2024

(30) Foreign Application Priority Data
Jun. 7, 2022 (EP) 22177579.4

(51) Int. Cl. G01N 33/50; G01N 33/574

(52) U.S. Cl. CPC G01N 33/5091; G01N 33/57426; G01N 2800/52

(57) ABSTRACT

The present invention concerns assessment of therapies for cancer and, in particular, hematopoietic cancers. In particular, it relates to a method for assessing and, preferably predicting response to a BCL-family inhibitor therapy in a subject suffering from cancer, preferably a hematopoietic cancer, comprising the steps of determining the amounts of the biomarkers BCL-2, BCL-xL, and MCL-1 in a tumor driving cell population, preferably leukemic stem cell (LSC) population, in a sample of said subject and comparing the amounts of the said biomarkers to a reference, whereby the response to a BCL-family inhibitor therapy is assessed. Furthermore, the present invention relates to a BCL-2 inhibitor, preferably Venetoclax, a BCL-xL and/or MCL-1 inhibitor, preferably Navitoclax, or a BCL-2 inhibitor in combination with at least one MCL-1 inhibitor or use in treating cancer, preferably a hematopoietic cancer, in a subject that has been assessed to benefit from a therapy using said inhibitors by using the method of the invention.

### CROSS-REFERENCE TO RELATED APPLICATIONS

This application is the U.S. National Stage of International Patent Application No. PCT/EP2023/054685, filed Feb. 24, 2023, which claims priority from European Patent Application No. 22177579.4, filed Jun. 7, 2022. The contents of these applications are incorporated herein by reference in their entirety.

The present invention concerns assessment of therapies for cancer and, in particular, hematopoietic cancers. In particular, it relates to a method for assessing and, preferably predicting response to a BCL-family inhibitor therapy in a subject suffering from cancer, preferably a hematopoietic cancer, comprising the steps of determining the amounts of the biomarkers BCL-2, BCL-xL, and MCL-1 in a tumor driving cell population preferably leukemic stem cell (LSC) population, in a sample of said subject and comparing the amounts of the said biomarkers to a reference, whereby the response to a BCL-family inhibitor therapy is assessed. Furthermore, the present invention relates to a BCL-2 inhibitor, preferably Venetoclax, a BCL-xL and/or MCL-I inhibitor, preferably Navitoclax, or a BCL-2 inhibitor in combination with at least one MCL-1 inhibitor or use in treating cancer, preferably a hematopoietic cancer, in a subject that has been assessed to benefit from a therapy using said inhibitors by using the method of the invention.

Acute myeloid leukemia (AML) remains a cancer with dismal prognosis, particularly in elderly or frail patients, ineligible for high dose chemotherapy as well as patients with high risk disease. The survival of AML cells is dependent on the expression of anti-apoptotic factors such as BCL-2.

In recent years, Venetoclax, a potent BCL-2 inhibitor (Konopleva et al., 2016), in combination with hypomethylating agents (HMAs) has replaced HMAs alone as standard of care treatment for AML patients unsuitable for intensive induction chemotherapy (DiNardo et al., 2020a). Moreover, Venetoclax has recently been added successfully to various high dose induction protocols, providing further evidence of its effectiveness in AML treatment beyond HMA combinations (DiNardo et al., 2021; Garcia et al., 2021).

HMAs in combination with Venetoclax are also currently being evaluated as first line treatment for adult AML patients eligible for intensive induction chemotherapy such as cytarabine and daunorubicin. Therefore, longitudinal studies linking treatment response to molecular- and cytogenetic aberrations are essential to identify the most suitable therapy and predict upfront resistance and relapse following initial response. The European leukemia network (ELN) risk classification currently used to guide treatment decisions for AML patients, was established based on data collected prior to Venetoclax-based treatment and might therefore not precisely predict response to HMA/Ventoclax (Cherry et al., 2021; Dohner et al., 2017). Several denominators for Venetoclax sensitivity have been proposed, such as cell of origin (Cai et al., 2020), apoptotic priming, (Bhatt et al., 2020) and monocytic differentiation of blasts (Cherry et al., 2021; Kuusanmaki et al., 2020: Pei et al., 2020). The latter has gained particular attention in several studies, and refers to AML samples previously classified as myelomonocytic (M4) or monocytic (M5) based on the French-American-British (FAB) Classification and/or that contain blast cells with high levels of CD11b+, CD64+ or CD68+ expression as detected by flow cytometry. Furthermore, ex vivo treatment and transcriptome data have suggested that monocytic AMLs represent a separate class of AMLs associated with high resistance HMA/Venetoclax treatment.

Additionally, a recent study claimed that this resistance can be observed in reactive oxygen species (ROS)-low, LSC-enriched AML populations of M4/5 patients (Pei et al., 2020). Importantly, dependence on MCL-1 rather than BCL-2 in LSCs from monocytic AMLs has been suggested to underlie the resilience against 5-AZA/Venetoclax. However, monocytic differentiation of AML was not associated with a worse patient outcome after treatment with HMA/Venetoclax in two recent independent clinical trials, failing to validate the hypothesis related to M4/5 AMLs above (DiNardo et al., 2020b; Stahl et al., 2021).

Thus, there is a need for assessing and predicting the response and efficacy to BCL-family inhibitor therapies such as Venetoclax therapies more reliably.

The technical problem underlying the present invention may be seen as the provision of means and methods for complying with the aforementioned need. The technical problem is solved by the embodiments characterized in the claims and herein below.

Therefore, the present invention relates to a method for assessing response to a BCL-family inhibitor therapy in a subject suffering from cancer, preferably a hematopoietic cancer comprising the steps of;
(a) determining the amounts of the biomarkers BCL-2, BCL-xL, and MCL-1 in a tumor driving cell population preferably leukemic stem cell (LSC) population in a sample of said subject; and
(b) comparing the amounts of the said biomarkers to a reference, whereby the response to a BCL-family inhibitor therapy is assessed.

It is to be understood that in the specification and in the claims, “a” or “an” can mean one or more of the items referred to in the following depending upon the context in which it is used. Thus, for example, reference to “an” item can mean that at least one item can be utilized.

As used in the following, the terms “have”, “comprise” or “include” are meant to have a non-limiting meaning or a limiting meaning. Thus, having a limiting meaning these terms may refer to a situation in which, besides the feature introduced by these terms, no other features are present in an embodiment described, i.e. the terms have a limiting meaning in the sense of “consisting of” or “essentially consisting of”. Having a non-limiting meaning, the terms refer to a situation where besides the feature introduced by these terms, one or more other features are present in an embodiment described.

Further, as used in the following, the terms “preferably”, “more preferably”, “most preferably”, “particularly”, “more particularly”, “typically”, and “more typically” are used in conjunction with features in order to indicate that these features are preferred features, i.e. the terms shall indicate that alternative features may also be envisaged in accordance with the invention.

Further, it will be understood that the term “at least one” as used herein means that one or more of the items referred to following the term may be used in accordance with the invention. For example, if the term indicates that at least one item shall be used this may be understood as one item or more than one item, i.e. two, three, four, five or any other number. Depending on the item the term refers to the skilled person understands as to what upper limit the term may refer, if any.

The term “about” in the context of the present invention means+/−20%, +/−10%, +/−5%, +/−2% or +/−1% from the indicated parameters or values. This also takes into account usual deviations caused by measurement techniques and the like.

The method of the present invention may encompass further steps prior to step a) or after step b) or between or within those steps. Typically, the method may include steps of pretreating the sample for the determination of the biomarkers prior to step a). Yet, the method may include steps such as recommending therapeutic measures after step b) on the basis of the assessment.

The term “assessing” as used herein refers to determining or predicting a response to a BCL-family inhibitor therapy in a subject suffering from a hematopoietic cancer. Accordingly, the said response may be either determined for a subject that received the therapy or it may be predicted for a subject prior to the onset of the said therapy. For a response prediction, it will be understood that the prediction shall be made within a predictive window. Typically, said window starts at the time point when the sample to be investigated by the method of the invention has been taken. The predictive window is, preferably within the range of at least one month, two months, three months, six months, nine months, one year, two years or three years. As will be understood by those skilled in the art, an assessment is usually not intended to be correct for 100% of the subjects to be investigated. The term, however, requires that the assessment is correct for a statistically significant portion of the subjects (e.g. a cohort in a cohort study). Whether a portion is statistically significant can be determined without further ado by the person skilled in the art using various well known statistic evaluation tools, e.g., determination of confidence intervals, p-value determination, Student's t-test, Mann-Whitney test etc. Details are found in Dowdy and Wearden, Statistics for Research. John Wiley & Sons, New York 1983. Preferred confidence intervals are at least 90%, at least 95%, at least 97%, at least 98% or at least 99%. The p-values are, preferably 0.1, 0.05, 0.01, 0.005, or 0.0001.

Preferably, said assessing response to a BCL-family inhibitor therapy comprises identifying whether a subject will benefit from the treatment by BCL-2 inhibitor, or not. Also preferably, said assessing response to a BCL-family inhibitor therapy comprises identifying whether a subject will benefit from the treatment by a BCL-xL and/or MCL-1 inhibitor, or not. Yet preferably, said assessing response to a BCL-family inhibitor therapy comprises identifying whether a subject will benefit from the treatment by BCL-2 inhibitor and at least one MCL-1 inhibitor, or not. As will be understood by those skilled in the art, the method of the present invention allows for identifying subjects that will benefit from the therapy (“rule-in”) or subjects that do not benefit from the therapy (“rule-out”) or both.

The term “BCL-family inhibitor” as used herein refers to an inhibitor that affects the biological function of a BCL-family member, preferably an anti-apoptotic BCL family member. Typically, such inhibitors bind to the anti-apoptotic BCL-family member and thereby affect its biological function, in particular, reduce or inhibit its biological activity. The anti-apoptotic BCL-family comprises several regulator proteins that are involved in the inhibition of apoptosis. Anti-apoptotic BCL-family members comprise BCL-2, BCL-xL, MCL-1, CED-9, Al, and Bfl-1. Of particular importance are BCL-2, BCL-xL and MCL-1. By reducing or inhibiting their activities, the BCL-family inhibitor, thus, allows that a cell can enter apoptotic cell death. The anti-apoptotic activity of BCL-family proteins is important for the development of many cancer entities which, due to said activity and other factors, become immortalized. Preferably, the BCL-family inhibitor referred to in accordance with the present invention is selected from the group consisting of: obatoclax, subatoclax, maritoclax, gossypol, apogossypol, TW-37, UMI-77, BDA-366, ABT-737, Navitoclax, Venetoclax, S64315 (MiK665), AZD5991, AMG176, AMG379, and ABBV467. More preferably, said BCL-family inhibitor is an inhibitor of BCL-2, preferably selected from the group consisting of: Venetoclax, and ABT-737, most preferably Venetoclax. Yet more preferably, said BCL-family inhibitor is an inhibitor of BCL-xL, preferably selected from the group consisting of: Navitoclax, ABT-737, A-1155463, and A-1331852, most preferably Navitoclax. Also more preferably, said BCL-family inhibitor is an inhibitor of MCL-1, preferably selected from the group consisting of: Navitoclax, S64315 (MIK665), AZD5991, AMG176, AMG379, and ABBV467, most preferably Navitoclax. Inhibitors such as Navitoclax may also inhibit other BCL-family members such as BCL-2 or MCL-1. However, Navitoclax is herein pivotally referred to as a BCL-xL inhibitor.

It will be understood that the BCL-family inhibitor referred to in accordance with the present invention may be used as a single drug therapy or it may be used in combination with other drugs. Preferably, the BCL-family inhibitor may be used in combination with an additional cancer treating agent such as a classic chemotherapy agent, more preferably a hypomethylating agent, preferably 5-azacytidine (5-AZA) decitabine or cytarabine, or an antibody such as Rituximab, or a target therapy agent such as Midostaurin.

The term “response to a BCL-family inhibitor therapy” as used herein refers to any response of a hematopoietic cancer or symptoms thereof which becomes clinically apparent in response to the BCL-family inhibitor therapy. Thus, the said response of the hematopoietic cancer may be beneficial for the subject suffering from the said cancer in that the cancer or symptoms thereof improve or it may be adverse to the subject in that no response is observable or the hematopoietic cancer or symptoms thereof worsens. Preferably, in accordance with the present invention a response to a BCL-family inhibitor therapy shall be any improvement of a hematopoietic cancer or symptoms thereof which becomes clinically apparent in response to the BCL-family inhibitor therapy from which the subject suffering from said cancer benefits.

The term “subject” as used herein relates to animals, preferably mammals, and, more preferably humans. The subject according to the present invention shall suffer from a hematopoietic cancer as described elsewhere herein.

The term “cancer” as used herein refers to hematopoietic cancer as well as to solid tumors such as colorectal cancer, pancreatic cancer, liver cancer, lung cancer, cancer of the nervous system and the like. The term cancer as used herein also encompasses premalignant cancer stages occurring in various cancer entities. Preferably, hematopoietic cancers may have premalignant stages such as myelodysplastic syndrome (MDS) which may develop into AML.

The term “hematopoietic cancer” as used herein refers to any cancer involving or affecting hematopoietic cells. The term also includes any kind of hematopoietic malignancy. Preferably, said hematopoietic cancer is acute myeloid leukemia (AML), B-, T-cell other lymphoma or leukemia or a plasma cell neoplasm.

The term “BCL-2” as used herein refers to the B-cell lymphoma 2 protein encoded in humans by the BCL-2 gene. It is the founding member of the Bcl-2 family of apoptosis regulator proteins. These proteins regulate apoptosis by either inhibiting apoptosis, i.e. being anti-apoptotic, or inducing it, i.e. being pro-apoptotic. The BCL-2 protein inhibit apoptosis and, thus, is anti-apoptotic. For BCL-2, there are two isoforms known in humans. Several orthologs of BCL-2 have been reported in various animal species. The BCL-2 protein is localized to the outer membrane of mitochondria, where it plays an important role in promoting cellular survival and inhibiting the actions of pro-apoptotic proteins. The pro-apoptotic proteins in the BCL-family such as Bax and Bak typically permeabilize mitochondrial membranes in order to release cytochrome C and ROS that are important signals in the apoptosis cascade. These pro-apoptotic proteins are in turn activated by BH3-only proteins, and are inhibited by the function of BCL-2 and its relative BCL-X1. In various cancer entities, the homeostatic balance between cell growth and cell death is impaired. The overexpression of the anti-apoptotic BCL-2 protein alone does not cause cancer. However, if the anti-apoptotic BCL-2 is overexpressed together with an oncogene simultaneously, cancer may result.

The BCL-2 protein referred to in accordance with the present invention is, preferably human BCL-2 having an amino acid sequence as deposited under UniProt accession number P10415 or mouse BCL-2 having an amino acid sequence as deposited under UniProt accession number P10417. It will be understood that the term “BCL-2” also relates to variants of said proteins. Such variants have at least the same essential biological and immunological properties as the aforementioned BCL-2 protein. In particular, they share the same essential biological and immunological properties if they are detectable by the same specific assays referred to in this specification. Moreover, it is to be understood that a variant as referred to in accordance with the present invention shall have an amino acid sequence which differs due to at least one amino acid substitution, deletion and/or addition wherein the amino acid sequence of the variant is still, preferably at least 50%, 60%, 70%, 80%, 85%, 90%, 92%, 95%, 97%, 98%, or 99% identical with the specific amino sequence of the human or mouse BCL-2 protein, preferably over the entire length of the said BCL-2 proteins, respectively.

The degree of identity between two amino acid sequences in accordance with the present invention can be determined by algorithms well known in the art. Preferably, the degree of identity is to be determined by comparing two optimally aligned sequences over a comparison window, where the fragment of amino acid sequence in the comparison window may comprise additions or deletions (e.g., gaps or overhangs) as compared to the reference sequence (which does not comprise additions or deletions) for optimal alignment. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm disclosed by Smith, by the homology alignment algorithm of Needleman, by the search for similarity method of Pearson, by computerized implementations of these algorithms (GAP, BESTFIT, BLAST, FAST, PASTA, and TFASTA in the Wisconsin Genetics Software Package, Genetics Computer Group (GCG), 575 Science Dr., Madison, WI) or by visual inspection. Given that two sequences have been identified for comparison, GAP and BESTFIT are preferably employed to determine their optimal alignment and, thus, the degree of identity. Preferably, the default values of 5.00 for gap weight and 0.30 for gap weight length are used. Variants referred to above may be allelic variants or any other species specific homologs, paralogs, or orthologs. Variants referred to above may be allelic variants or any other species specific homologs, paralogs, or orthologs.

The term “BCL-xL” as used herein refers to the B-cell lymphoma extra-large protein encoded in humans by the BCL-2-like 1 gene. Like BCL-2, it is a member of the Bcl-2 family of apoptosis regulator proteins and inhibits apoptosis, i.e. anti-apoptotic. Several orthologs of BCL-xL have been reported in various animal species. The BCL-xL protein is a transmembrane protein in the outer mitochondrial membrane, where it plays a role in promoting cellular survival and inhibiting the actions of pro-apoptotic proteins. Like BCL-2, BCL-xL has been reported to be involved due to its anti-apoptotic activity in the development of various cancers.

The BCL-xL protein referred to in accordance with the present invention is, preferably human BCL-xL having an amino acid sequence as deposited under UniProt accession number Q07817 or mouse BCL-xL having an amino acid sequence as deposited under UniProt accession number Q64373. It will be understood that the term “BCL-xL” also relates to variants of said proteins. Such variants have at least the same essential biological and immunological properties as the aforementioned BCL-xL protein. In particular, they share the same essential biological and immunological properties if they are detectable by the same specific assays referred to in this specification. Moreover, it is to be understood that a variant as referred to in accordance with the present invention shall have an amino acid sequence which differs due to at least one amino acid substitution, deletion and/or addition wherein the amino acid sequence of the variant is still, preferably at least 50%, 60%, 70%, 80%, 85%, 90%. 92%, 95%, 97%, 98%, or 99% identical with the specific amino sequence of the human or mouse BCL-xL protein, preferably over the entire length of the said BCL-xL proteins, respectively.

The term “MCL-1” as used herein refers to the induced myeloid leukemia cell differentiation protein which is a protein that in humans is encoded by the MCL-1 gene. Like BCL-2, it is an anti-apoptotically acting member of the Bcl-2 family of apoptosis regulator. It acts in the outer mitochondrial membrane similar like BCL-2 or BCL-xL. In humans, two isoforms have been reported. Moreover, several orthologs of MCL-1 have been reported in various animal species. The MCL-1 protein plays a role in promoting cellular survival and inhibiting the actions of pro-apoptotic proteins. MCL-1 has been reported to be involved due to its anti-apoptotic activity in the development of various cancers, too.

The MCL-1 protein referred to in accordance with the present invention is, preferably human MCL-1 having an amino acid sequence as deposited under UniProt accession number Q07820 or mouse MCL-1 having an amino acid sequence as deposited under UniProt accession number P97287. It will be understood that the term “MCL-1” also relates to variants of said proteins. Such variants have at least the same essential biological and immunological properties as the aforementioned MCL-1 protein. In particular, they share the same essential biological and immunological properties if they are detectable by the same specific assays referred to in this specification. Moreover, it is to be understood that a variant as referred to in accordance with the present invention shall have an amino acid sequence which differs due to at least one amino acid substitution, deletion and/or addition wherein the amino acid sequence of the variant is still, preferably at least 50%, 60%, 70%, 80%, 85%, 90%, 92%, 95%, 97%, 98%, or 99% identical with the specific amino sequence of the human or mouse MCL-1 protein, preferably over the entire length of the said MCL-1 proteins, respectively.

The term “biomarker” as used in accordance with the present invention relates to the biomarker protein or any precursor protein, fragment or derivative thereof which is naturally generated and which reflects the amount of the biomarker protein. Moreover, the term also encompasses any nucleic acid molecule which reflects the amount of biomarker protein. Preferably, such transcribed nucleic acid molecules are the messenger RNA molecules (mRNA) or any precursor or variant thereof, including pre-mRNA or mRNA for splice variants. Those RNA nucleic acid molecules may be determined as biomarkers in accordance with the present invention as well. Thus, it will be understood that if, e.g., BCL-2 shall be determined as biomarker in accordance with the present invention, either BCL-2 protein may be determined or a transcribed nucleic acid molecule encoding BCL-2 protein such as BCL-2 mRNA. The same applies for BCL-xL and MCL-1 as biomarkers as well as for all other biomarkers referred to herein, except specified otherwise.

The term “tumor driving cell population” as used herein refers to any cell population in said subject which represent a reservoir for the development of further cancer cells of the cancer and, preferably the hematopoietic cancer. The tumor-driving cell population, preferably is not a population of quiescent cancer cells but rather consists of cancer cells that have long-term self-renewal potential and thus can divide unlimitedly starting from a single cell and are contributing to the tumor development such as frequently dividing cancer cells and/or cancer cells with metastasizing potential. Thus, the said population may consist of cancer cells or cancer cell precursors. Preferably, the tumor driving cell population is a leukemic stem cell (LSC) population sometimes also referred to herein as LSC-like population. Preferably, said LSC population is characterized by increased expression of at least one biomarker selected from the group consisting of: GPR56. CD34 and BCL-2. Typically, the LSC population is characterized by increased expression of at least two or at least three of the aforementioned biomarkers. Most preferably, the LSC population is a population of cells expression all of the aforementioned biomarkers. More preferably, said expression is increased compared to the expression of the at least one biomarker in monocyte-like AML cells. LSC populations useful in accordance with the present invention may be determined by either determining the expression of at least one of the aforementioned biomarkers or by determining the expression of at least one, preferably at least 5, of the biomarkers shown in the Table 1, below. The expression of those biomarkers has been shown to correlate with GPR56 expression in LSCs. Typically, said amounts of the said biomarkers are determined quantification of RNA expression, preferably PCR-based techniques, or specific antibody-based quantification, preferably by flow cytometry techniques, Western blots or immunofluorescence measurements. The skilled person is well aware of how those techniques can be applied. Preferably, the individual cells comprised in the sample are investigated for the expression of the aforementioned biomarker(s) characteristic for the tumor driving cell population, preferably the LSC population and—at the same time—for the expression of BCL-2, BCL-xL and MCL-1 to be used for the assessment carried out in accordance with the method of the present invention. Thus, particular preferred in accordance with the present invention are methods which allow the simultaneous measurement of all of these biomarkers in individual cells such as flow cytometry or single-cell real time PCR techniques. When using such techniques, advantageously, there is no requirement for a physical separation of the tumor driving cell population from the sample. The identification of the said population and the determination of the expression levels for BCL-2, BCL-xL and MCL-1 can be made in a digital environment after the measurement.

TABLE 1
Biomarkers the expression of which correlates with GPR56 expression in LSCs

| | | | | | | |
|---|---|---|---|---|---|---|
| p_val | avg_log2FC | pct. 1 | pct. 2 | p_val_adj | cluster | gene |

A) Proteins

| | | | | | | | |
|---|---|---|---|---|---|---|---|
| GPR56-TotalA | 6.66E−247 | 1.087684874 | 1 | 0.91 | 2.53E−245 | GPR56Pos | GPR56-TotalA |
| CD34-TotalA | 2.93E−160 | 0.752611226 | 1 | 0.957 | 1.11E−158 | GPR56Pos | CD34-TotalA |
| CD7-TotalA | 4.44E−140 | 0.77211877 | 1 | 0.995 | 1.69E−138 | GPR56Pos | CD7-TotalA |
| CD99-TotalA | 2.92E−121 | 0.297346054 | 1 | 1 | 1.11E−119 | GPR56Pos | CD99-TotalA |
| CD25-TotalA | 2.25E−95 | 0.464512944 | 0.971 | 0.843 | 8.56E−94 | GPR56Pos | CD25-TotalA |
| CD117-TotalA | 3.00E−84 | 0.560452329 | 0.998 | 0.841 | 1.14E−82 | GPR56Pos | CD117-TotalA |
| CD123-TotalA | 2.14E−62 | 0.28906356 | 1 | 0.998 | 8.14E−61 | GPR56Pos | CD123-TotalA |

B) RNAs

| | | | | | | | |
|---|---|---|---|---|---|---|---|
| BAALC | 1.08E−189 | 1.56723227 | 0.762 | 0.123 | 1.81E−185 | GPR56Pos | BAALC |
| IGHM | 5.55E−186 | 2.642999132 | 0.764 | 0.149 | 9.30E−182 | GPR56Pos | IGHM |
| FAM30A | 3.74E−172 | 2.090161747 | 0.769 | 0.169 | 6.27E−168 | GPR56Pos | FAM30A |
| SPINK2 | 6.10E−170 | 2.01633924 | 0.988 | 0.625 | 1.02E−165 | GPR56Pos | SPINK2 |
| HOPX | 2.27E−137 | 1.885896257 | 0.831 | 0.295 | 3.81E−133 | GPR56Pos | HOPX |
| SMIM24 | 2.43E−129 | 1.319473643 | 0.793 | 0.229 | 4.07E−125 | GPR56Pos | SMIM24 |
| C1QTNF4 | 2.15E−124 | 1.600805476 | 0.647 | 0.134 | 3.61E−120 | GPR56Pos | C1QTNF4 |
| DNTT | 2.25E−121 | 1.185953397 | 0.438 | 0.039 | 3.77E−117 | GPR56Pos | DNTT |
| ADGRG1 | 3.09E−118 | 0.602777813 | 0.426 | 0.036 | 5.18E−114 | GPR56Pos | ADGRG1 |
| CD7 | 3.01E−116 | 1.268903961 | 0.833 | 0.312 | 5.04E−112 | GPR56Pos | CD7 |
| AKR1C3 | 2.11E−114 | 1.203755111 | 0.758 | 0.25 | 3.54E−110 | GPR56Pos | AKR1C3 |
| GNAI1 | 1.28E−110 | 0.915751457 | 0.636 | 0.152 | 2.15E−106 | GPR56Pos | GNAI1 |
| CTSW | 4.94E−103 | 1.005055077 | 0.988 | 0.772 | 8.29E−99 | GPR56Pos | CTSW |
| CD96 | 3.36E−97 | 0.928917331 | 0.756 | 0.247 | 5.63E−93 | GPR56Pos | CD96 |
| PDLIM1 | 1.36E−96 | 0.997917258 | 0.957 | 0.673 | 2.28E−92 | GPR56Pos | PDLIM1 |
| NPM1 | 1.66E−95 | 0.829889068 | 1 | 0.995 | 2.78E−91 | GPR56Pos | NPM1 |
| TRBC2 | 1.21E−92 | 0.710678512 | 0.384 | 0.045 | 2.02E−88 | GPR56Pos | TRBC2 |
| CD99 | 5.02E−87 | 1.143198796 | 0.996 | 0.987 | 8.42E−83 | GPR56Pos | CD99 |
| RPS5 | 1.11E−85 | 0.612194409 | 1 | 1 | 1.87E−81 | GPR56Pos | RPS5 |
| SEPTIN6 | 8.25E−80 | 0.818436018 | 0.971 | 0.783 | 1.38E−75 | GPR56Pos | SEPTIN6 |
| LIME1 | 7.95E−79 | 0.887319263 | 0.55 | 0.146 | 1.33E−74 | GPR56Pos | LIME1 |
| RPL3 | 9.14E−79 | 0.503427989 | 1 | 1 | 1.53E−74 | GPR56Pos | RPL3 |
| NPR3 | 4.43E−77 | 0.664755105 | 0.519 | 0.128 | 7.42E−73 | GPR56Pos | NPR3 |
| CDK6 | 1.36E−76 | 0.868335526 | 0.952 | 0.709 | 2.27E−72 | GPR56Pos | CDK6 |
| MZB1 | 1.04E−75 | 0.716847389 | 0.403 | 0.07 | 1.74E−71 | GPR56Pos | MZB1 |
| STMN1 | 1.21E−73 | 1.121629072 | 0.971 | 0.697 | 2.03E−69 | GPR56Pos | STMN1 |
| AC243960.1 | 1.15E−72 | 0.700738566 | 0.585 | 0.185 | 1.93E−68 | GPR56Pos | AC243960.1 |
| EEF1B2 | 7.30E−72 | 0.629066622 | 1 | 1 | 1.22E−67 | GPR56Pos | EEF1B2 |
| SOX4 | 2.34E−71 | 0.779673638 | 0.994 | 0.902 | 3.93E−67 | GPR56Pos | SOX4 |
| SPNS3 | 1.83E−70 | 0.780966566 | 0.843 | 0.432 | 3.06E−66 | GPR56Pos | SPNS3 |
| ITM2C | 5.89E−70 | 0.770966483 | 0.961 | 0.716 | 9.87E−66 | GPR56Pos | ITM2C |
| LNCAROD | 1.18E−69 | 0.572576743 | 0.473 | 0.116 | 1.98E−65 | GPR56Pos | LNCAROD |
| CD34 | 5.75E−69 | 0.583586858 | 0.37 | 0.065 | 9.65E−65 | GPR56Pos | CD34 |
| HNRNPA1 | 1.78E−68 | 0.588575715 | 1 | 1 | 2.98E−64 | GPR56Pos | HNRNPA1 |
| ACTG1 | 2.49E−68 | 0.654362777 | 1 | 1 | 4.17E−64 | GPR56Pos | ACTG1 |
| ADA | 4.95E−67 | 0.703681381 | 0.599 | 0.211 | 8.29E−63 | GPR56Pos | ADA |
| CD9 | 6.91E−67 | 1.106416859 | 0.802 | 0.478 | 1.16E−62 | GPR56Pos | CD9 |
| OCIAD2 | 2.46E−64 | 0.656197297 | 0.593 | 0.214 | 4.13E−60 | GPR56Pos | OCIAD2 |
| DSTN | 2.93E−64 | 1.011827144 | 0.915 | 0.705 | 4.91E−60 | GPR56Pos | DSTN |
| SSBP2 | 1.64E−63 | 0.817359589 | 0.824 | 0.473 | 2.76E−59 | GPR56Pos | SSBP2 |
| ZNF22 | 2.85E−63 | 0.678004046 | 0.843 | 0.431 | 4.78E−59 | GPR56Pos | ZNF22 |
| EGFL7 | 3.72E−63 | 0.822247344 | 0.647 | 0.241 | 6.24E−59 | GPR56Pos | EGFL7 |
| CD79A | 1.01E−62 | 0.573856319 | 0.376 | 0.078 | 1.69E−58 | GPR56Pos | CD79A |
| MPG | 1.38E−62 | 0.679401621 | 0.977 | 0.872 | 2.31E−58 | GPR56Pos | MPG |
| LAT2 | 8.77E−62 | 0.698108954 | 0.946 | 0.79 | 1.47E−57 | GPR56Pos | LAT2 |
| TUBB | 1.44E−61 | 0.952188055 | 0.948 | 0.728 | 2.42E−57 | GPR56Pos | TUBB |
| TP53INP1 | 1.81E−60 | 1.185108375 | 0.506 | 0.169 | 3.04E−56 | GPR56Pos | TP53INP1 |
| ACY3 | 5.02E−60 | 0.677235715 | 0.44 | 0.113 | 8.42E−56 | GPR56Pos | ACY3 |
| ITM2A | 7.09E−60 | 0.62464924 | 0.702 | 0.287 | 1.19E−55 | GPR56Pos | ITM2A |
| LDHA | 3.21E−57 | 0.62387774 | 0.998 | 0.986 | 5.38E−53 | GPR56Pos | LDHA |
| TSTD1 | 1.05E−56 | 0.638504214 | 0.773 | 0.385 | 1.77E−52 | GPR56Pos | TSTD1 |
| RPSA | 1.05E−54 | 0.50101589 | 1 | 0.999 | 1.75E−50 | GPR56Pos | RPSA |
| NUDT8 | 1.28E−53 | 0.59516916 | 0.533 | 0.191 | 2.15E−49 | GPR56Pos | NUDT8 |
| ARMH1 | 2.35E−52 | 0.629740631 | 0.965 | 0.754 | 3.93E−48 | GPR56Pos | ARMH1 |
| C5orf56 | 6.37E−51 | 0.50331107 | 0.502 | 0.177 | 1.07E−46 | GPR56Pos | C5orf56 |
| IL2RG | 1.63E−50 | 0.643950092 | 0.946 | 0.757 | 2.74E−46 | GPR56Pos | IL2RG |
| IGFBP7 | 2.24E−50 | 0.556495659 | 0.996 | 0.948 | 3.75E−46 | GPR56Pos | IGFBP7 |
| SOD1 | 4.09E−50 | 0.589278346 | 0.981 | 0.946 | 6.86E−46 | GPR56Pos | SOD1 |
| RAB37 | 1.82E−49 | 0.568267665 | 0.541 | 0.201 | 3.05E−45 | GPR56Pos | RAB37 |
| BLNK | 3.21E−49 | 0.555097308 | 0.395 | 0.111 | 5.39E−45 | GPR56Pos | BLNK |
| TSC22D1 | 3.51E−49 | 1.176797855 | 0.721 | 0.427 | 5.89E−45 | GPR56Pos | TSC22D1 |
| IKZF1 | 4.52E−49 | 0.646663631 | 0.977 | 0.889 | 7.58E−45 | GPR56Pos | IKZF1 |
| NUDT5 | 6.97E−49 | 0.673601108 | 0.818 | 0.554 | 1.17E−44 | GPR56Pos | NUDT5 |
| PEBP1 | 1.60E−48 | 0.590665939 | 0.99 | 0.935 | 2.69E−44 | GPR56Pos | PEBP1 |
| IMPDH2 | 5.13E−47 | 0.720830459 | 0.833 | 0.584 | 8.61E−43 | GPR56Pos | IMPDH2 |
| MATK | 6.01E−47 | 0.518240011 | 0.533 | 0.203 | 1.01E−42 | GPR56Pos | MATK |
| PHB | 3.13E−46 | 0.586712973 | 0.926 | 0.798 | 5.24E−42 | GPR56Pos | PHB |
| FHL1 | 3.48E−45 | 0.549268982 | 0.595 | 0.269 | 5.84E−41 | GPR56Pos | FHL1 |
| IL3RA | 8.72E−45 | 0.564198193 | 0.543 | 0.226 | 1.46E−40 | GPR56Pos | IL3RA |
| AFF3 | 9.75E−45 | 0.66480627 | 0.793 | 0.47 | 1.63E−40 | GPR56Pos | AFF3 |
| YWHAQ | 2.54E−44 | 0.528079894 | 0.948 | 0.847 | 4.26E−40 | GPR56Pos | YWHAQ |
| LDHB | 4.25E−44 | 0.540334079 | 1 | 0.983 | 7.12E−40 | GPR56Pos | LDHB |
| HSP90AB1 | 6.66E−44 | 0.533701962 | 1 | 0.998 | 1.12E−39 | GPR56Pos | HSP90AB1 |
| SMIM3 | 1.86E−43 | 0.690763804 | 0.913 | 0.721 | 3.12E−39 | GPR56Pos | SMIM3 |
| KIF2A | 3.06E−43 | 0.570274091 | 0.853 | 0.593 | 5.13E−39 | GPR56Pos | KIF2A |
| ESD | 1.23E−42 | 0.574817839 | 0.855 | 0.612 | 2.06E−38 | GPR56Pos | ESD |
| CD47 | 2.05E−42 | 0.527875839 | 0.942 | 0.789 | 3.43E−38 | GPR56Pos | CD47 |
| RNF125 | 5.26E−42 | 0.577832214 | 0.634 | 0.315 | 8.82E−38 | GPR56Pos | RNF125 |
| RASD1 | 8.99E−42 | 0.577777814 | 0.362 | 0.105 | 1.51E−37 | GPR56Pos | RASD1 |
| PRDX1 | 1.94E−40 | 0.682318248 | 0.959 | 0.909 | 3.25E−36 | GPR56Pos | PRDX1 |
| DDAH2 | 4.32E−40 | 0.629317423 | 0.868 | 0.665 | 7.24E−36 | GPR56Pos | DDAH2 |
| CD81 | 6.79E−40 | 0.580603158 | 0.752 | 0.439 | 1.14E−35 | GPR56Pos | CD81 |
| CD69 | 9.22E−40 | 0.934892785 | 0.779 | 0.508 | 1.55E−35 | GPR56Pos | CD69 |
| AGPS | 1.91E−39 | 0.594816222 | 0.636 | 0.338 | 3.21E−35 | GPR56Pos | AGPS |
| PSIP1 | 6.58E−39 | 0.565985285 | 0.915 | 0.684 | 1.10E−34 | GPR56Pos | PSIP1 |
| RHOH | 7.76E−39 | 0.51216109 | 0.564 | 0.252 | 1.30E−34 | GPR56Pos | RHOH |
| MPC2 | 2.22E−38 | 0.527186169 | 0.876 | 0.673 | 3.73E−34 | GPR56Pos | MPC2 |
| BIN1 | 2.48E−38 | 0.68091265 | 0.68 | 0.377 | 4.16E−34 | GPR56Pos | BIN1 |
| SLC38A1 | 4.43E−38 | 0.511833907 | 0.862 | 0.593 | 7.43E−34 | GPR56Pos | SLC38A1 |
| GAMT | 6.30E−38 | 0.536998496 | 0.605 | 0.3 | 1.06E−33 | GPR56Pos | GAMT |
| TRG-AS1 | 3.13E−37 | 0.590593045 | 0.733 | 0.457 | 5.25E−33 | GPR56Pos | TRG-AS1 |
| TUBA1B | 3.93E−37 | 0.775453579 | 1 | 0.973 | 6.59E−33 | GPR56Pos | TUBA1B |
| IFI16 | 5.43E−37 | 0.608155574 | 0.804 | 0.55 | 9.11E−33 | GPR56Pos | IFI16 |
| TCF4 | 6.67E−37 | 0.538871649 | 0.595 | 0.3 | 1.12E−32 | GPR56Pos | TCF4 |
| LSP1 | 8.92E−37 | 0.530490656 | 0.973 | 0.831 | 1.50E−32 | GPR56Pos | LSP1 |
| NREP | 9.50E−37 | 0.649086867 | 0.696 | 0.415 | 1.59E−32 | GPR56Pos | NREP |
| CCT2 | 3.28E−36 | 0.568175164 | 0.853 | 0.649 | 5.50E−32 | GPR56Pos | CCT2 |
| SMARCB1 | 4.14E−36 | 0.543744709 | 0.754 | 0.49 | 6.93E−32 | GPR56Pos | SMARCB1 |
| TUBA1A | 5.86E−36 | 0.625802482 | 0.967 | 0.896 | 9.82E−32 | GPR56Pos | TUBA1A |
| HMGA1 | 6.36E−36 | 0.599073448 | 0.855 | 0.655 | 1.07E−31 | GPR56Pos | HMGA1 |
| PARP1 | 1.24E−34 | 0.591149228 | 0.781 | 0.573 | 2.08E−30 | GPR56Pos | PARP1 |
| CCT7 | 1.78E−33 | 0.541423407 | 0.872 | 0.699 | 2.99E−29 | GPR56Pos | CCT7 |
| IDH2 | 1.93E−33 | 0.588183977 | 0.802 | 0.606 | 3.23E−29 | GPR56Pos | IDH2 |
| CCT3 | 1.52E−32 | 0.529977419 | 0.87 | 0.741 | 2.54E−28 | GPR56Pos | CCT3 |
| NPW | 2.45E−32 | 0.515373043 | 0.512 | 0.232 | 4.10E−28 | GPR56Pos | NPW |
| WASF2 | 4.40E−32 | 0.500476809 | 0.919 | 0.863 | 7.37E−28 | GPR56Pos | WASF2 |
| RUNX2 | 1.07E−31 | 0.574207575 | 0.645 | 0.363 | 1.79E−27 | GPR56Pos | RUNX2 |
| UBE2J1 | 1.13E−31 | 0.563022333 | 0.878 | 0.721 | 1.89E−27 | GPR56Pos | UBE2J1 |
| SPRY1 | 1.99E−30 | 0.562295762 | 0.291 | 0.091 | 3.34E−26 | GPR56Pos | SPRY1 |
| ARPC5L | 2.06E−30 | 0.574194556 | 0.824 | 0.658 | 3.46E−26 | GPR56Pos | ARPC5L |
| GYPC | 4.53E−30 | 0.502969932 | 0.767 | 0.509 | 7.59E−26 | GPR56Pos | GYPC |
| LDLRAD4 | 1.11E−29 | 0.52520852 | 0.494 | 0.243 | 1.86E−25 | GPR56Pos | LDLRAD4 |
| TYMS | 1.50E−29 | 0.737894714 | 0.517 | 0.245 | 2.52E−25 | GPR56Pos | TYMS |
| PIK3R1 | 1.14E−28 | 0.546942252 | 0.969 | 0.916 | 1.91E−24 | GPR56Pos | PIK3R1 |
| SNHG7 | 1.41E−28 | 0.741438202 | 0.888 | 0.786 | 2.36E−24 | GPR56Pos | SNHG7 |
| ATF7IP2 | 1.73E−28 | 0.522442687 | 0.769 | 0.553 | 2.89E−24 | GPR56Pos | ATF7IP2 |
| SESN1 | 2.82E−28 | 0.622191185 | 0.804 | 0.646 | 4.73E−24 | GPR56Pos | SESN1 |
| SMARCA2 | 4.52E−28 | 0.535782985 | 0.899 | 0.78 | 7.58E−24 | GPR56Pos | SMARCA2 |
| YWHAH | 5.23E−28 | 0.544854818 | 0.975 | 0.949 | 8.77E−24 | GPR56Pos | YWHAH |
| TFPI | 2.89E−27 | 0.503481909 | 0.618 | 0.352 | 4.85E−23 | GPR56Pos | TFPI |
| AMD1 | 3.24E−27 | 0.505657598 | 0.897 | 0.74 | 5.43E−23 | GPR56Pos | AMD1 |
| SMC3 | 2.36E−26 | 0.502858327 | 0.816 | 0.619 | 3.95E−22 | GPR56Pos | SMC3 |
| CARHSP1 | 5.68E−25 | 0.507242001 | 0.688 | 0.471 | 9.52E−21 | GPR56Pos | CARHSP1 |
| CDK4 | 1.28E−24 | 0.504102556 | 0.663 | 0.431 | 2.15E−20 | GPR56Pos | CDK4 |
| PAICS | 1.42E−24 | 0.542554018 | 0.574 | 0.349 | 2.38E−20 | GPR56Pos | PAICS |
| SLC9A3R2 | 2.04E−24 | 0.526583666 | 0.277 | 0.1 | 3.42E−20 | GPR56Pos | SLC9A3R2 |
| RAN | 1.25E−23 | 0.555191879 | 0.95 | 0.913 | 2.10E−19 | GPR56Pos | RAN |
| PIK3IP1 | 5.30E−23 | 0.589095267 | 0.669 | 0.473 | 8.89E−19 | GPR56Pos | PIK3IP1 |
| MCM7 | 1.89E−22 | 0.530489422 | 0.508 | 0.283 | 3.17E−18 | GPR56Pos | MCM7 |
| PCLAF | 4.35E−21 | 0.637492692 | 0.533 | 0.308 | 7.30E−17 | GPR56Pos | PCLAF |
| DCTPP1 | 7.79E−19 | 0.507877932 | 0.661 | 0.467 | 1.31E−14 | GPR56Pos | DCTPP1 |
| HIST1H4C | 1.69E−18 | 0.577356281 | 0.847 | 0.715 | 2.84E−14 | GPR56Pos | HIST1H4C |
| TSC22D3 | 1.21E−11 | 0.527980029 | 0.998 | 0.997 | 2.02E−07 | GPR56Pos | TSC22D3 |
| KLF2 | 2.64E−08 | 1.01213593 | 0.897 | 0.924 | 0.000442998 | GPR56Pos | KLF2 |

The term “sample” refers to a sample of a body fluid, to a sample of separated cells or to a sample from a tissue or an organ comprising or suspect to comprise the tumor driving cell population. Samples of body fluids can be obtained by well-known techniques and include, preferably samples of blood. Tissue or organ samples, such as bone marrow samples, may be obtained by, e.g., biopsy. Separated cells may be obtained from the body fluids or the tissues or organs by separating techniques such as centrifugation or cell sorting.

Determining the amount of one or more biomarker(s) as referred to in accordance with the present invention encompasses measuring the amount or concentration, preferably semi-quantitatively or quantitatively.

Measuring carried out directly or indirectly. Direct measuring relates to measuring the amount or concentration of the biomarker based on a signal which is obtained from the biomarker molecule itself and the intensity of which directly correlates with the number of molecules of the biomarker present in the sample. Such a signal—sometimes referred to herein as intensity signal—may be obtained, e.g., by measuring an intensity value of a specific physical or chemical property of the biomarker molecule. Indirect measuring includes measuring of a signal obtained from a secondary component, i.e. a component not being the biomarker molecule itself.

In accordance with the present invention, determining the amount of a biomarker can be achieved by all known means for determining such amounts in a sample. Said means comprise immunoassay devices and methods which may utilize labeled molecules in various sandwich, competition, or other assay formats. Said assays will develop a signal which is indicative for the presence or absence of the peptide or polypeptide. Moreover, the signal strength can, preferably be correlated directly or indirectly (e.g. reverse-proportional) to the amount of the biomarker present in a sample. Further suitable methods comprise measuring a physical or chemical property specific for the biomarkers. Said methods comprise, preferably biosensors, optical devices coupled to immunoassays, biochips, or other analytical devices such as chromatography devices or single cell analyzing devices such as FACS analyzers or devices for single cell PCR analysis.

Preferably, the biomarker(s) to be determined in accordance with the present invention may be determined as protein. To this end, typically a binding molecule is applied that specifically binds to the said biomarker protein and that can be detected either by a detectable label present in the binding molecule or by a secondary binding molecule that specifically binds to the first binding molecule and comprises a detectable label.

A binding molecule as referred to in this context may be any molecule that is capable of specifically binding to the biomarker to be detected. Preferably, such a binding molecule may be an antibody or an antibody mimetic or an aptamer.

An antibody in accordance with the present invention may encompass all types of antibodies which specifically bind to the biomarker protein. Preferably, the antibody of the present invention is a monoclonal antibody, a polyclonal antibody, a single chain antibody, a chimeric antibody or any fragment or derivative of such antibodies being still capable of binding to the biomarker protein specifically. Such fragments and derivatives comprised by the term antibody as used herein encompass a bispecific antibody, a synthetic antibody, a Fab, F(ab)2 Fv or scFv fragment, or a chemically modified derivative of any of these antibodies. Specific binding as used in the context of the anti-body of the present invention means that the antibody does not cross react with other molecules present in the sample to be investigated. Specific binding can be tested by vanous well-known techniques. Antibodies or fragments thereof, in general, can be obtained by using methods which are described in standard text books, e.g., in Harlow and Lane “Antibodies, A Laboratory Manual”, CSH Press, Cold Spring Harbor, 1988. Monoclonal antibodies can be prepared by the techniques which comprise the fusion of mouse myeloma cells to spleen cells derived from immunized mammals and, preferably immunized mice. Preferably, an immunogenic peptide is applied to a mammal. The said peptide is, preferably conjugated to a carrier protein, such as bovine serum albumin. thyroglobulin, and keyhole limpet hemocyanin (KLH). Depending on the host species, various adjuvants can be used to increase the immunological response. Such adjuvants encompass, preferably Freund's adjuvant, mineral gels, e.g., aluminum hydroxide, and surface-active substances, e.g., lysolecithin, pluronic polyols, polyanions, peptides, oil emulsions, keyhole limpet hemocyanin, and dinitrophenol. Mono-clonal antibodies which specifically bind to an analyte can be subsequently prepared using the well-known hybridoma technique, the human B cell hybridoma technique, and the EBV hybridoma technique. Detection systems using antibodies are based on the highly specific binding affinity of antibodies for a specific antigen, i.e. the biomarker protein. Binding events result in a physicochemical change that can be detected as described elsewhere herein.

An antibody mimetic in accordance with the present invention encompasses peptide or protein molecules that have antibody-like binding properties but which are not structurally related to antibodies. Such antibody mimetics have typically a molecular weight of up to 20 kDa. Preferably, an antibody mimetic in accordance with the present invention may be an affibody molecule, an affilin, an affimer, an affitin, an alphabody, an anticalin, an avimer, an DARPin, a fynomer, a gastrobody, a Kunitz domain protein, a monobody, a nanoCLAMP, a repebody, a centryn or an obody.

An aptamer according to the present invention may be a nucleic acid or peptide aptamer. Specific aptamers can be generated by techniques well known in the art including, e.g., the systematic evolution of ligands by exponential enrichment (SELEX) technology. Peptide aptamers comprise of a variable peptide loop attached at both ends to a protein scaffold. This double structural constraint shall increase the binding affinity of the peptide aptamer into the nano-molar range. Said variable peptide loop length is, typically, composed of ten to twenty amino acids, and the scaffold may be any protein having improved solubility and compacity properties, such as thioredoxin-A. Peptide aptamer selection can be made using different systems including, e.g., the yeast two-hybrid system. The term also encompasses optimized or modified aptamers such as optimers, split aptamers or X-aptamers.

A detectable label as referred to herein which may be used in accordance with the invention include gold particles, latex beads, acridan ester, luminol, ruthenium, enzymatically active labels, radioactive labels, magnetic labels, e.g., magnetic beads, including paramagnetic and superparamagnetic labels, and fluorescent labels. Enzymatically active labels include e.g. horseradish peroxidase, alkaline phosphatase, beta-Galactosidase, Luciferase, and derivatives thereof. Suitable substrates for detection include di-amino-benzidine (DAB), 3,3′-5,5′-tetramethylbenzidine, NBT-BCIP (4-nitro blue tetrazolium chloride and 5-bromo-4-chloro-3-indolyl-phosphate. A suitable enzyme-substrate combination may result in a colored reaction product, fluorescence or chemiluminescence, which can be measured according to methods known in the art (e.g. using a light-sensitive film or a suitable camera system). As for measuring the enzymatic reaction, the criteria given above apply analogously. Typical fluorescent labels include fluorescent proteins (such as GFP and its derivatives), Cy3, Cy5. Texas Red, Fluorescein, and the Alexa dyes. Also the use of quantum dots as fluorescent labels is contemplated. Typical radioactive labels include 35S, 1251, 32P, 33P and the like. A radioactive label can be detected by any method known and appropriate, e.g. a light-sensitive film or a phosphor imager. Suitable labels may also be or comprise tags, such as biotin, digoxygenin, His-, GST-, FLAG-, GFP-, MYC-tag, influenza A virus haemagglutinin (HA), maltose binding protein, and the like.

Also preferably, the biomarker(s) to be determined in accordance with the present invention may be determined as nucleic acid molecules, preferably as transcripts such as mRNAs. If a transcript encoding a biomarker protein is to be detected, it will be understood that, typically, a nucleic acid molecule being either RNA or DNA may be used for detection as detecting agent according to the invention.

A nucleic acid molecule useful as a detection agent in accordance with the present invention refers to DNA or RNA molecules that are capable of specifically interacting with the transcript for the biomarker. The biomarker transcript is a nucleic acid molecule, too, and specific binding can be achieved via the specific interactions of complementary or reverse complementary nucleotide strands. Typically, the nucleic acid useful as detection agent is selected from the group consisting of: an antisense RNA, a ribozyme, a siRNA or a micro RNA. Also preferably, oligonucleotides having complementary and reverse complementary sequences may be used as target transcript specific primers for PCR-based detection techniques.

An antisense RNA as used herein refers to RNA which comprise a nucleic acid sequence which is essentially or perfectly complementary to the target transcript. Typically, an antisense nucleic acid molecule essentially consists of a nucleic acid sequence being complementary to at least 100 contiguous nucleotides, more preferably at least 200, at least 300, at least 400 or at least 500 contiguous nucleotides of the target transcript. How to generate and use antisense nucleic acid molecules is well known in the art.

A ribozyme as used herein refers to catalytic RNA molecules possessing a well-defined tertiary structure that allows for specific binding to target RNA and catalyzing either the hydrolysis of one of their own phosphodiester bonds (self-cleaving ribozymes), or the hydrolysis of bonds in target RNAs, but they have also been found to catalyze the aminotransferase activity of the ribosome. How to generate and use such ribozymes is well known in the art.

A siRNA as used herein refers to small interfering RNAs (siRNAs) which are complementary to target RNAs (encoding a gene of interest) and diminish or abolish gene expression by RNA interference (RNAi). RNAi is generally used to silence expression of a gene of interest by targeting mRNA. Briefly, the process of RNAi in the cell is initiated by double stranded RNAs (dsRNAs) which are cleaved by a ribonuclease, thus producing siRNA duplexes. The siRNA binds to another intracellular enzyme complex which is thereby activated to target whatever mRNA molecules are homologous (or complementary) to the siRNA sequence. The function of the complex is to target the homologous mRNA molecule through base pairing interactions between one of the siRNA strands and the target mRNA. Thus, siRNA molecules are capable of specific binding and can be used as detection agents according to the present invention.

microRNA as used herein refers to a self-complementary single-stranded RNA which comprises a sense and an antisense strand linked via a hairpin structure. The micro RNA comprise a strand which is complementary to an RNA targeting sequences comprised by a transcript to be downregulated, micro RNAs are processed into smaller single stranded RNAs and, therefore, presumably also act via the RNAi mechanisms. How to design and to synthesize microRNAs which specifically bind and degrade a transcript of interest is known in the art. Due to the specific nucleic acid binding capabilities, they can be used as detection agents according to the invention.

Detection systems using nucleic acid as detection molecules can be based on complementary base pairing interactions. The recognition process is based on the principle of complementary nucleic acid base pairing. If the target nucleic acid sequence is known, complementary sequences can be synthesized and labeled for detection. The hybridization event can be detected by known measures. Moreover, using PCR-based techniques, even low amounts of transcripts can be determined and quantified. How to carry out such PCR-based techniques is well known to the skilled artisan.

The term “amount” as used herein encompasses the absolute amount of the biomarker, the relative amount or concentration of the biomarker as well as any value or parameter which correlates thereto or can be derived therefrom. Such values or parameters comprise intensity signal values from all specific physical or chemical properties obtained from the said biomarker or a detection molecule and/or detectable label. It is to be understood that values correlating to the aforementioned amounts or parameters can also be obtained and/or modified by all standard mathematical operations.

Typically, the amount as referred to herein may be a normalized amount, i.e. an amount which is calculated based on the measured value for the amount of the biomarker and a normalizing parameter that allows for correction of physiological variability between different samples and/or technical variability caused by technical differences or irregularities between different measurements. Preferably, normalization for the measured amounts of the individual biomarkers in a test sample shall be made using measurements for said biomarkers in a one or more normalization sample(s) or a normalization value calculated on such measurements. Preferably, the median amounts for the biomarkers determined in a plurality of normalization samples obtained from subjects responding to the BCL-family inhibitor therapy is used as a normalization value in accordance with the present invention. The plurality of normalization samples used for deriving the median, preferably comprises at least 10, at least 100 or at least 1,000 samples. Moreover, the normalization may also take into account general expression levels in the sample by correcting the measured amounts of the biomarker by a measured amount of one or more housekeeping proteins, such as IgG.

The determined amounts of the biomarkers BCL-2. BCL-xL, and MCL-1 are compared to a reference in accordance with the method of the present invention.

The term “reference” as used in accordance with the present invention relates to any amount or value which by comparison to the determined amount of the biomarker allows for assessing the response of the subject to the BCL-family inhibitor therapy. Thus, the reference amount or value may be obtained from a subject or a group of subjects known to respond to the BCL-family inhibitor therapy. In such a case, if similar amounts for the biomarkers are present in the test sample, the tested subject shall be assessed as a responder as well whereas if amounts for the biomarkers which differ from the reference are determined in the test sample, the subject may be assessed as a non-responder. The reference amount or value may also be obtained from a subject or a group of subjects known to not respond to the BCL-family inhibitor therapy (non-responder). In such a case, if similar amounts for the biomarkers are present in the test sample, the tested subject shall be assessed as a non-responder as well whereas if amounts for the biomarkers which differ from the reference are determined in the test sample, the subject may be assessed as a responder.

Moreover, the reference may also be a score integrating reference amounts or values derived therefrom for different biomarkers. Preferably, a score to be used in accordance with the present invention integrates the amounts for BCL-2, BCL-xL and MCL-1. This allows to consider possible escape mechanisms for specific BCL-family inhibitors. For example, high amount of BCL-xL and MCL-1 and low BCL-2 amounts in cells of tumor driving cell population of a subject may drive an escape mechanism for a BCL-2 inhibitor therapy and, thus, that the subject is a non-responder. Contrary, high amounts of BCL-2 and low amounts of BCL-xL and MCL-1 indicate that the BCL-2 inhibitor will act efficiently and, thus, that the subject is a responder. Considering the potential escape pathways for BCL-family inhibitors, it has been found that a score integrating the three components BCL-2, BCL-xL and MCL-1 is helpful for assessing the response to the BCL-family inhibitor therapy, specifically in LSC-like cells. The score, preferably envisaged in accordance with the present invention is a response score, also referred to herein as prediction score, being the ratio of the determined amount of BCL-2 and the combined determined amounts of BCL-xL and MCL-1.

Preferably, a response score for assessing the response to a BCL-2 inhibitor, preferably Venetoclax, in the subject to be investigated by the method of the present invention may be preferably calculated as follows:

$$Response score = [ determined amount of BCL - 2 ] / ( [ determined amount of BCL - xL ] + [ determined amount of MCL - 1 ] )$$

Preferably, a response score for assessing the response to a BCL-xL inhibitor, preferably Navitoclax, in the subject to be investigated by the method of the present invention may be preferably calculated as follows:

$$Response score = 0.5 ( [ determined amount of BCL - 2 ] + [ determined amount of BCL - xL ] ) / [ determined amount of MCL - 1 ]$$

Preferably, a response score for assessing the response to a MCL-1 inhibitor in the subject to be investigated by the method of the present invention may be preferably calculated as follows:

$$Response score = 0.5 ( [ determined amount of MCL - 1 ] + [ determined amount of BCL - 2 ] ) / [ determined amount of BCL - xL ]$$

As discussed elsewhere herein, the determined amounts for the biomarkers are, preferably normalized amounts.

More preferably, a reference for said response score may be a reference score calculated as described before, wherein said reference is derived from non-responder population. More preferably, said reference is between about 0.6 and about 1.0, preferably is about 0.8.

More preferably, the method of the present invention comprises determining a response score for a BCL-2 inhibitor, a BCL-xL inhibitor and a MCL-1 inhibitor as described above. Most preferably, the three response scores are compared with each other and evaluated. For the evaluation, the three determined response scores may be compared and the highest response score shall be indicative for a beneficial BCL-family inhibitor therapy. For example, if the BCL-2 response score is highest among the three scores, a BCL-2 inhibitor therapy (e.g., Venetoclax therapy) is beneficial, if the BCL-xL MCL-1 response score is highest among the three scores, a BCL-xL inhibitor therapy (e.g., Navitoclax therapy) is beneficial and if the MCL-1-score is the highest, a BCL-2 inhibitor and a MCL-1 inhibitor therapy shall be beneficial. Thereby, the assessment can be further improved.

Based on the comparison the response to a BCL-family inhibitor therapy is assessed. Preferably, said assessing comprises identifying whether the subject tested by the method of the present invention is a responder or non-responder to the BCL-family inhibitor therapy. Further, the assessment allows for identifying whether a subject benefits from the therapy in case of being a responder, or not in case of being a non-responder. The term “comparing” as used herein encompasses comparing the amount(s), value(s) or response score as defined above. The comparison referred to in step (b) of the method of the present invention may be carried out manually or computer assisted. If the step is carried out in a computer-assisted format, an assessment may also be made automatically on the basis of the result of the comparison. Preferably, the computer program carrying out the said assessment will provide the result of the assessment in a suitable output format.

In a further step, the method may also make recommendations with respect to the BCL-family inhibition therapy, i.e. it may recommend to continue or discontinue or to change the therapy. Using an expert system and a suitable database with recommendations, such as a relational database having allocated possible recommendations to assessments, the step of making recommendations may be carried out in a computer-implemented form as well.

Advantageously, it was confirmed in the studies underlying the present invention that AMLs are assessed at bulk level, monocytic AMLs were dominated by monocytic blasts which conferred resistance to ex vivo 5-AZA/Venetoclax treatment. Conflictingly, a retrospective analysis of 54 AML patients receiving HMA/Venetoclax as frontline treatment at Heidelberg university hospital did not show a prognostic value of myelo-/monocytic differentiation. It was shown that monocytic blast subpopulations, although insensitive to 5-AZA/Venetoclax due to MCL-1 rather than BCL-2 dependence, do not have significant functional LSC-potential. This suggests that other leukemic cells must play a role to explain the clinical data, that a population of tumor driving cells and, in particular an LSC population of immature, GPR56+ stem-like cells, present in all investigated AML specimens irrespective of their overall phenotypic presentation may be used for assessing responsiveness to BCL-family inhibitor therapy. GPR56+ stem-like cells are highly enriched for BCL-2-dependent, functional LSCs and rapidly cleared in AML patients responsive to 5-AZA/Venetoclax therapy demonstrating the effectiveness of Venetoclax on LSCs in general. In accordance with the findings of the present invention, a response score was established based on BCL-2, BCL-xL and MCL-1 expression levels in GPR56+ LSCs, which identifies subjects that benefit from BCL-family inhibitor therapies. Moreover, the method established can be easily implemented in clinical flow cytometry facilities.

Thanks to the present invention, and the method for assessing BCL-family inhibitor therapy, it is possible to:
decide between standard chemotherapy and BCL-family inhibitor therapy i.e. in first line;
make a risk stratification in elderly patients that need to decide whether they want to be subjected to chemotherapy at all;
decide on a second line treatment using intensive chemotherapy in a patient that does not respond to standard chemotherapy
decide between different members of the BCL-family inhibitors as therapeutics.

All explanations and definitions of the terms made above apply mutatis mutandis for the following embodiments.

In a preferred embodiment of the method of the present invention, said assessing response to a BCL-family inhibitor therapy comprises identifying whether a subject will benefit from the treatment by a BCL-2 inhibitor, preferably Venetoclax, or not.

More preferably, said comparing of the said biomarkers to a reference comprises calculating the ratio of the amount of BCL-2 to the combined amounts of BCL-xL and MCL-1 in order to obtain a prediction score and comparing said prediction score to a reference. Preferably, a prediction score larger than the reference is indicative for a subject that will benefit from the treatment by the BCL-2 inhibitor whereas a predictive score lower than the reference is indicative for a subject that will not benefit from the treatment by the BCL-2 inhibitor. More preferably, said reference is a reference value derived from non-responder population, most preferably said reference is between about 0.6 and about 1.0, preferably is about 0.8.

In another preferred embodiment of the method of the present invention, said assessing response to a BCL-family inhibitor therapy comprises identifying whether a subject will benefit from the treatment by a BCL-xL and/or at least one MCL-1 inhibitor, preferably Navitoclax, or not.

More preferably, said comparing of the said biomarkers to a reference comprises calculating the ratio of the half of the combined amounts of BCL-2 and BCL-xL to the amount of MCL-1 in order to obtain a prediction score and comparing said prediction score to a reference. Preferably, a prediction score larger than the reference is indicative for a subject that will benefit from the treatment by a BCL-xL and/or MCL-1 inhibitor, whereas a predictive score lower than the reference is indicative for a subject that will not benefit from the treatment by a BCL-xL and/or MCL-1 inhibitor. More preferably, said reference is a reference value derived from non-responder population, most preferably said reference is between about 0.6 and about 1.0, preferably is about 0.8.

In yet a preferred embodiment of the method of the present invention, said assessing response to a BCL-family inhibitor therapy comprises identifying whether a subject will benefit from the treatment by a BCL-2 inhibitor and at least one MCL-1 inhibitor, preferably Venetoclax, and said at least one MCL-1 inhibitor is AZD5991 or MIK665, or not.

More preferably, said comparing of the said biomarkers to a reference comprises calculating the ratio of the amount of BCL-2 to the combined amounts of BCL-xL and MCL-1 in order to obtain a prediction score and comparing said prediction score to a reference. Preferably, a prediction score larger than the reference is indicative for a subject that will benefit from the treatment by a BCL-2 inhibitor and at least one MCL-1 inhibitor, whereas a predictive score lower than the reference is indicative for a subject that will not benefit from the treatment by a BCL-2 inhibitor and at least one MCL-1 inhibitor. More preferably, said reference is a reference value derived from non-responder population, most preferably said reference is between about 0.6 and about 1.0, preferably is about 0.8.

The present invention further contemplates a BCL-2 inhibitor, preferably Venetoclax for use in treating cancer, preferably a hematopoietic cancer, in a subject that has been assessed to benefit from therapy using said BCL-2 inhibitor by a method of the invention.

The term “treating” as used herein refers to ameliorating and/or curing a disease as referred to herein, preventing progression of the disease or at least an amelioration of at least one symptom associated with the said disease. It will be understood that a treatment as referred to herein will, in all likelihood, not be successful in all subjects which received the treatment. However, it is envisaged that the treatment is effective in at least a statistically significant portion of the subjects that are treated. Whether a statistically significant portion, e.g., of a cohort of subjects, can be successfully treated may, preferably be determined, e.g., by statistical tests discussed elsewhere herein in more detail.

The present invention further relates to a BCL-xL and/or MCL-1 inhibitor, preferably Navitoclax, for use in treating cancer, preferably a hematopoietic cancer in a subject that has been assessed to benefit from therapy using said BCL-xL and/or MCL-1 inhibitor by a method of the invention.

The present invention relates to a BCL-2 inhibitor, preferably Venetoclax, in combination with at least one MCL-1 inhibitor for use in treating cancer, preferably a hematopoietic cancer in a subject that has been assessed to benefit from therapy using said BCL-2 inhibitor in combination with at least one MCL-1 inhibitor by a method of the invention.

Moreover, the present invention also relates to a method for treating a subject suffering from cancer, preferably hematopoietic cancer by a BCL-family inhibitor therapy, said method comprises assessing the response to the BCL-family inhibitor therapy for said subject by carrying out the method of the invention and, administering a BCL-family inhibitor to said subject if the subject is assessed to benefit from said therapy in a therapeutically effective amount.

A therapeutically effective amount refers to an amount of the BCL-family inhibitor to be used which prevents, ameliorates or treats the symptoms accompanying a disease or condition referred to in this specification. Therapeutic efficacy and toxicity of the compound can be determined by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is the therapeutic index, and it can be expressed as the ratio, LD₅₀/ED₅₀. The dosage regimen will be determined by the attending physician and other clinical factors. As is well known in the medical arts, dosages for any one patient depends upon many factors, including the patient's size, body surface area, age, the particular compound to be administered, sex, time and route of administration, general health, and other drugs being administered concurrently. Progress can be monitored by periodic assessment. Dosage and dosage regimen for the BCL-family inhibitors according to the present invention are, preferably those known in the art.

In a preferred embodiment of the aforementioned method for treating a subject, said BCL-family inhibitor is a BCL-2 inhibitor, preferably Venetoclax, a BCL-xL and/or MCL-1 inhibitor, preferably Navitoclax, or a BCL-2 inhibitor, preferably Venetoclax, in combination with at least one MCL-1 inhibitor.

The present invention also relates to a device for assessing response to a BCL-family inhibitor therapy in a subject suffering from cancer, preferably a hematopoietic cancer comprising:
(a) an analyzing unit capable of determining the amounts of the biomarkers BCL-2, BCL-xL, and MCL-1 in a tumor driving cell population preferably leukemic stem cell (LSC) population in a sample of said subject; and
(b) an evaluation unit comprising a data processor capable of comparing the amounts of the said biomarkers to a reference, whereby the response to a BCL-family inhibitor therapy is assessed.

The term “device” as used herein refers to a system comprising the aforementioned units operatively linked to each other as to allow the determination of the presence, absence or abundance of biomarkers and evaluation thereof according to the method of the invention such that an assessment can be provided.

The analyzing unit, typically, comprises at least one detection element being capable of detecting the biomarkers present in the sample. Prior to introducing the sample intro the detection element, the sample may be pre-treated by detection molecules in order to generated detectable signals, e.g., by allowing the formation of biomarker-antibody complexes whereby the antibody as detection molecule comprises a detectable label that can be detected by the detection element. The detection element may also comprise a reaction zone that allows carrying out a chemical detection reaction such as a PCR. The detection element shall be adapted to determine the amount of the biomarkers. The determined amount can be subsequently transmitted to the evaluation unit.

The evaluation unit comprises a data processing element, such as a computer, with an implemented algorithm for determining the amount of biomarkers present in the sample. The processing unit as referred to in accordance with the method of the present invention, typically, comprises a Central Processing Unit (CPU) and/or one or more Graphics Processing Units (GPUs) and/or one or more Application Specific Integrated Circuits (ASICs) and/or one or more Tensor Processing Units (TPUs) and/or one or more field-programmable gate arrays (FPGAs) or the like. A data processing element may be a general purpose computer or a portable computing device, for example. It should also be understood that multiple computing devices may be used together, such as over a network or other methods of transferring data, for performing one or more steps of the methods disclosed herein. Exemplary computing devices include desk-top computers, laptop computers, personal data assistants (“PDA”), cellular devices, smart or mobile devices, tablet computers, servers, and the like. In general, a data processing element comprises a processor capable of executing a plurality of instructions (such as a program of software). The evaluation unit, typically, comprises or has access to a memory. A memory is a computer readable medium and may comprise a single storage device or multiple storage devices, located either locally with the computing device or accessible to the computing device across a network, for example. Computer-readable media may be any available media that can be accessed by the computing device and includes both volatile and non-volatile media. Further, computer readable-media may be one or both of removable and non-removable media. By way of example, and not limitation, computer-readable media may comprise computer storage media. Exemplary computer storage media includes, but is not limited to, RAM, ROM, EEPROM, flash memory or any other memory technology. CD-ROM, Digital Versatile Disk (DVD) or other optical disk storage, magnetic cassettes, magnetic tape, magnetic disk storage or other magnetic storage devices, or any other medium which can be used for storing a plurality of instructions capable of being accessed by the computing device and executed by the processor of the computing device. The evaluation unit may also comprise or has access to an output device. Exemplary output devices include fax machines, displays, printers, and files, for example. According to some embodiments of the present disclosure, a computing device may perform one or more steps of a method disclosed herein, and thereafter provide an output, via an output device, relating to a result, indication, ratio or other factor of the method.

Preferably, said device is adopted to carry out the method of the present invention.

The present invention, furthermore, relates to a kit for assessing response to a BCL-family inhibitor therapy in a subject suffering from cancer, preferably a hematopoietic cancer comprising detection molecules for determining the amounts of the biomarkers BCL-2, BCL-xL, and MCL-1 in a tumor driving cell population preferably leukemic stem cell (LSC) population in a sample of said subject.

The term “kit” as used herein refers to collection of the aforementioned components, typically, provided in separately or within a single container. The container also typically comprises instructions for carrying out the method of the present invention. These instructions may be in the form of a manual or may be provided by a computer program code which is capable of carrying out or supports the determination of the biomarkers referred to in the methods of the present invention when implemented on a computer or a data processing device. The computer program code may be provided on a data storage medium or device such as an optical storage medium (e.g., a Compact Disc) or directly on a computer or data processing device or may be provided in a download format such as a link to an accessible server or cloud. Moreover, the kit may usually comprise standards for reference amounts of biomarkers for calibration purposes as described elsewhere herein in detail. The kit according to the present invention may also comprise further components which are necessary for carrying out the method of the invention such as solvents, buffers, washing solutions and/or reagents required for detection of the released second molecule. Further, it may comprise the device of the invention either in parts or in its entirety.

The following embodiments are particular preferred embodiments according to the invention.

Embodiment 1: A method for assessing response to a BCL-family inhibitor therapy in a subject suffering from cancer, preferably a hematopoietic cancer, comprising the steps of:
(a) determining the amounts of the biomarkers BCL-2, BCL-xL, and MCL-1 in a tumor driving cell population, preferably leukemic stem cell (LSC) population, in a sample of said subject; and
(b) comparing the amounts of the said biomarkers to a reference, whereby the response to a BCL-family inhibitor therapy is assessed.

Embodiment 2: The method of embodiment 1, wherein said hematopoietic cancer is acute myeloid leukemia (AML), B-, T-cell other lymphoma or leukemia or a plasma cell neoplasm.

Embodiment 3: The method of embodiment 1 or 2, wherein said assessing response to a BCL-family inhibitor therapy comprises identifying whether a subject will benefit from the treatment by BCL-2 inhibitor, or not.

Embodiment 4: The method of embodiment 3, wherein said comparing of the said biomarkers to a reference comprises calculating the ratio of the amount of BCL-2 to the combined amounts of BCL-xL and MCL-1 in order to obtain a prediction score and comparing said prediction score to a reference.

Embodiment 5: The method of embodiment 4, wherein a prediction score larger than the reference is indicative for a subject that will benefit from the treatment by the BCL-2 inhibitor.

Embodiment 6: The method of embodiment 4, wherein a predictive score lower than the reference is indicative for a subject that will not benefit from the treatment by the BCL-2 inhibitor.

Embodiment 7: The method of any one of embodiments 4 to 6, wherein said reference is a reference value derived from non-responder population.

Embodiment 8: The method of embodiment 7, wherein said reference is between about 0.6 and about 1.0, preferably is about 0.8 Embodiment 9: The method of any one of embodiments 3 to 8, wherein said BCL-2 inhibitor is Venetoclax.

Embodiment 10: The method of embodiment 9, wherein said Venetoclax is used in combination with an additional cancer treating agent such as a classic chemotherapy agent, more preferably a hypomethylating agent, preferably 5-azacytidine (5-AZA), decitabine or cytarabine, or an antibody such as Rituximab, or a target therapy agent such as Midostaurin.

Embodiment 11: The method of embodiment 1 or 2, wherein said assessing response to a BCL-family inhibitor therapy comprises identifying whether a subject will benefit from the treatment by a BCL-xL and/or MCL-1 inhibitor, or not.

Embodiment 12: The method of embodiment 11, wherein said comparing of the said biomarkers to a reference comprises calculating the ratio of the half of the combined amounts of BCL-2 and BCL-xL to the amount of MCL-1 in order to obtain a prediction score and comparing said prediction score to a reference.

Embodiment 13: The method of embodiment 12, wherein a prediction score larger than the reference is indicative for a subject that will benefit from the treatment by a BCL-xL and/or MCL-1 inhibitor.

Embodiment 14: The method of embodiment 12, wherein a predictive score lower than the reference is indicative for a subject that will not benefit from the treatment by a BCL-xL and/or MCL-1 inhibitor.

Embodiment 15: The method of embodiment 12 or 14, wherein said reference is a reference value derived from non-responder population.

Embodiment 16: The method of embodiment 15, wherein said reference is between about 0.6 and about 1.0, preferably is about 0.8.

Embodiment 17: The method of any one of embodiments 11 to 16, wherein said BCL-xL and/or MCL-1 inhibitor is Navitoclax.

Embodiment 18: The method of embodiment 1 or 2, wherein said assessing response to a BCL-family inhibitor therapy comprises identifying whether a subject will benefit from the treatment by BCL-2 inhibitor and at least one MCL-1 inhibitor, or not.

Embodiment 19: The method of embodiment 18, wherein said comparing of the said biomarkers to a reference comprises calculating the ratio of the amount of BCL-2 to the combined amounts 25 of BCL-xL and MCL-1 in order to obtain a prediction score and comparing said prediction score to a reference.

Embodiment 20: The method of embodiment 19, wherein a prediction score larger than the reference is indicative for a subject that will benefit from the treatment by a BCL-2 inhibitor and at least one MCL-1 inhibitor.

Embodiment 21: The method of embodiment 19, wherein a predictive score lower than the reference is indicative for a subject that will not benefit from the treatment by a BCL-2 inhibitor and at least one MCL-1 inhibitor.

Embodiment 22: The method of embodiment 19 or 21, wherein said reference is a reference value derived from non-responder population.

Embodiment 23: The method of embodiment 22, wherein said reference is between about 0.6 and about 1.0, preferably is about 0.8.

Embodiment 24: The method of any one of embodiments 18 to 23, wherein said BCL-2 inhibitor is Venetoclax and said at least one MCL-1 inhibitor is AZD5991 or MIK665.

Embodiment 25: The method of any one of embodiments 1 to 24, wherein said LSC population is characterized by increased expression of at least one biomarker selected from the group consisting of: GPR56, CD34 and BCL-2.

Embodiment 26: The method of embodiment 25, wherein said expression is increased compared to the expression of the at least one biomarker in monocyte-like AML cells.

Embodiment 27: The method of any one of embodiments 1 to 26, wherein said amounts of the said biomarkers are determined quantification of RNA expression or specific antibody-based quantification, preferably by flow cytometry techniques.

Embodiment 28: A BCL-2 inhibitor, preferably Venetoclax for use in treating cancer, preferably a hematopoietic cancer, in a subject that has been assessed to benefit from therapy using said BCL-2 inhibitor by a method of any one of embodiments 3 to 10 and 24 to 27.

Embodiment 29: A BCL-xL and/or MCL-1 inhibitor, preferably Navitoclax, for use in treating cancer, preferably a hematopoietic cancer, in a subject that has been assessed to benefit from therapy using said BCL-xL and/or MCL-1 inhibitor by a method of any one of embodiments 11 to 17 and 24 to 27.

Embodiment 30: A BCL-2 inhibitor, preferably Venetoclax, in combination with at least one MCL-1 inhibitor for use in treating cancer, preferably a hematopoietic cancer, in a subject that has been assessed to benefit from therapy using said BCL-2 inhibitor in combination with at least one MCL-1 inhibitor by a method of any one of embodiments 18 to 27.

Embodiment 31: A method for treating a subject suffering from cancer, preferably hematopoietic cancer, by a BCL-family inhibitor therapy, said method comprises assessing the response to the BCL-family inhibitor therapy for said subject by carrying out the method of the invention and, administering a BCL-family inhibitor to said subject if the subject is assessed to benefit from said therapy.

Embodiment 32: The method of embodiment 31, wherein said BCL-family inhibitor is a BCL-2 inhibitor, preferably Venetoclax, a BCL-xL and/or MCL-1 inhibitor, preferably Navitoclax, or a BCL-2 inhibitor, preferably Venetoclax, in combination with at least one MCL-1 inhibitor.

Embodiment 33: A device for assessing response to a BCL-family inhibitor therapy in a subject suffering from cancer, preferably a hematopoietic cancer, comprising:
(a) an analyzing unit capable of determining the amounts of the biomarkers BCL-2, BCL-xL, and MCL-1 in a tumor driving cell population, preferably leukemic stem cell (LSC) population, in a sample of said subject; and
(b) an evaluation unit comprising a data processor capable of comparing the amounts of the said biomarkers to a reference, whereby the response to a BCL-family inhibitor therapy is assessed.

Embodiment 34: A kit for assessing response to a BCL-family inhibitor therapy in a subject suffering from cancer, preferably a hematopoietic cancer, comprising detection molecules for determining the amounts of the biomarkers BCL-2. BCL-xL, and MCL-1 in a tumor driving cell population preferably leukemic stem cell (LSC) population in a sample of said subject.

All references cited throughout the specification are herewith incorporated by reference with respect to the specifically mentioned disclosure content as well as in their entireties.

### FIGURES

FIG. 1: Monocytic characteristics of AML predict ex vivo response but not clinical response to 5-AZAN % EN.
A) 24 AML cell lines classified as primitive (Prim-AML, N=15) or monocytic (Mono-AML, N=9) based on CD64 surface expression (Mono-AML: MFI >3500, Prim-AML: MFI<1000) and were treated ex vivo with 1.5 μM of 5-AZA and increasing concentrations of VEN for 72 hours. Representative data of two independent replicates. Mean±SEM of technical replicates.
B) Mononuclear cells (MNCs) of diagnostic AML patient samples (N=12) were treated ex vivo for 72 h on a drug matrix with increasing 5-AZA and VEN concentrations. Unsupervised clustering was performed based on viability. Each quadrat represents one well with a specific 5-AZA/VEN combination on the drug matrix. C and D) Patient characteristics of 54 patients previously untreated for AML treated with 5-AZA/VEN in the first line were retrospectively assessed for risk factors of refractoriness to therapy. Univariant logistic regression was performed for every parameter. Multivariant logistic was performed on parameters with p<0.1 in the univariant analysis. E) Pre-treatment percentage of CD64⁺ CD11b⁺ AML cells among total AML bulk from cryostored samples of 41 first-line HMA/VEN patients. Each dot represents an individual AML patient with the line marking the mean. Mann-Whitney test was used to compare the two groups.

FIG. 2: Functional and transcriptomic LSC-like cells are predominantly located in GPR56⁺ immature fraction not CD64′CD11b⁺ Mature fraction.
A) FACS gating strategy for Mature, Non-LSC-like and LSC-Iike populations. Displayed are AML bulk from primitive CD34⁺ (NPM1-wildtype), CD34⁻ (NPM1-mutated) and monocytic sample (NPM1-mutated). B) Percentages of Mature. Non-LSC-like and LSC-like populations among bulk AML cells in 72 diagnostic AML samples sorted by frequency of Mature population. C) Schematic overview of experimental setup for xenotransplantation experiment and RNA-sequencing of FACS-sorted subpopulations. D) Percentage of human engrafiment obtained from Mature, Non-LSC and LSC-like subpopulations of 14 AML samples at endpoints in bone marrow of NSG mice. Each dot represents an individual mouse with the line marking the mean engraftment. E) Mean percentage of human engraftment per mouse obtained from Mature, Non-LSC and LSC-like subpopulations of 14 AML samples at endpoints in bone marrow of NSG mice. Each dot represents an individual AML patient with the line marking the mean engraftment. Friedmann test was used to compare LSC-like with Non-LSC-like and Mature populations. F) PCA plot of bulk RNA-seq data from LSC-like and Mature subpopulations from Prim-AMLs (N=11) and Mono-AMLs (N=6) annotated based on population and AML class. Each dot represents a population from an AML sample. G) LSC17 score in LSC-like and Mature subpopulations from Prim-AML (N=11) or Mono-AML (N=6) patient samples. LSC17 score was calculated for each AML sample as the mean expression of the 17 LSC signature genes. H, I and J) Normalized counts of H BCL-2, I MCL-1 and J BCL-2L1 in LSC-like and Mature subpopulations from Prim-AML (N=11) or Mono-AML (N=6) patient samples. AML bulk is defined as MNCs from AML patients after exclusion of dead cells, doublets, lymphocytes and nucleated erythrocytes. K) Schematic representation of intracellular staining to measure BCL-2, MCL-1 and BCL-xL by flow cytometry. L and M) Mean Fluorescence Intensity (MFI) of BCL-2 in L AML bulk and M LSC-like and Mature subpopulations from Prim-AML (N=11) or Mono-AML (N=7) patient samples. N and O) Mean Fluorescence Intensity (MFI) of MCL-1 in N AML bulk and O LSC-like and Mature subpopulations from Prim-AML (N=11) or Mono-AML (N=7) patient samples. P and Q) Representative tSNE plots of P HIAML46 (Prim-AML) and Q HIAML33 (Mono-AML) highlighting expression of CD64, CD34, GPR56, MCL-1, BCL-2 and BCL-xL. Two-Way ANOVA with Tukey correction for multiple comparisons test was used to compared groups of four and Mann-Whitney test was used to compare groups of two unless specified otherwise. Each dot represents an AML patient sample with the line marking the mean unless specified otherwise.

FIG. 3: LSC-like and Mature subpopulations show distinct apoptotic dependencies and response to 5-AZA/VEN therapy independent of AML class.
A) Workflow for C—F. MNCs of diagnostic AML patient samples were stained with surface antibodies, followed by BH3 profiling and quantification of area under the curve (AUC) to assess apoptotic susceptibility in bulk and sub-gated populations. B) Overview of assessed BH3 mimetics and their target proteins. C and D) AUC of VEN mediated Cytochrome-C release in C AML bulk and D LSC-like and Mature subpopulations from Prim-AML (N=11) or Mono-AML (N=7) patient samples. E and F) AUC of MS1 mediated Cytochrome-C release in E AML bulk and F LSC-like and Mature subpopulations from Prim-AML (N=11) or Mono-AML (N=7) patient samples. Each dot represents an individual AML patient sample with the line marking the mean. Mann-Whitney test was used to compare groups of two and Two-Way ANOVA with Tukey correction for multiple comparisons test was used to compared groups of four. G) Schematic representation of ex vivo treatment strategy for H-J. MNCs of diagnostic AML patient samples (N=17) were treated ex vivo for 24 hours at 1.5 μM 5-AZA and 100 nM VEN. H) Relative viability of LSC-like and Mature subpopulations from Prim-AML (N=11) or Mono-AML (N=7) patient samples was compared using Two-Way ANOVA with Tukey correction for multiple comparisons test. I and J) Representative tSNE plots of I HIAML46 (Prim-AML) and J HIAML33 (Mono-AML) highlighting expression of CD64 and GPR56 in 5-AZA/VEN treated and untreated controls. K) Schematic representation of PBMC collection strategy of AML patients undergoing 5-AZAVEN therapy. L) Quantification of Mature and LSC-like cell counts from PBMCs relative to pre-therapy in the first week of 5-AZA/VEN treatment in 3 patients undergoing therapy initiation. Each dot on the same line represents an individual timepoint of the same patient. M) Representative gating strategy highlighting population dynamics of LSC-like and Mature AML cell frequencies during the first week of 5-AZA/VEN treatment. N) Schematic representation of ex vivo treatment strategy for O—P. MNCs of HMA/VEN first line treated AML patients (N=26) were treated ex vivo for 24 hours at 1.5 μM 5-AZA and 100 nM VEN. O—P) Relative viability of LSC-like and Mature subpopulations were compared using Mann-Whitney test. Each dot represents an AML patient sample with the line marking the mean unless specified otherwise.

FIG. 4: Response to HMA/VEN therapy in AML can be predicted using BCL-2 family protein levels in LSC-like cells.
A) Schematic representation of experimental design for B-J. Response score was calculated based on normalized BCL-2 family protein expression levels in LSC-like cells from diagnostic AML patients receiving first line HMA/VEN therapy with known outcomes. B) Response score of AML patients from cohort 1 and HMA/VEN therapy outcome. C) Duration of HMA/VEN therapy represented as treatment cycles of AML patients from cohort 1 with above (>0.4) and below (<0.4) median response score. D) Response score of AML patients from cohort 2 and HMA/VEN therapy outcome. E) Duration of HMA/VEN therapy represented as treatment cycles of AML patients from cohort 2 with above (>0.4) and below (<0.4) median response score. F) Response score of AML patients from both cohorts combined and HMA/VEN therapy outcome. G) Receiver operating characteristic curve of response score and therapy outcomes of both cohorts. H) Duration of HMA/VEN therapy represented as event-free survival of AML patients from both cohorts combined with above (>0.4) and below (<0.4) median response score. I) Duration of HMA/VEN therapy represented as event-free survival of AML patients who achieve complete remission from both cohorts combined with above (>0.4) and below (<0.4) median response score. J) Patient characteristics of both cohorts with retrospectively assessed risk factors of refractoriness to therapy. Univariant logistic regression was performed for every parameter. Multivariant logistic was performed on parameters with p<0.15 in the univariant analysis. K) Schematic representation of experimental design for L-M. Response score was calculated based on normalized BCL-2 family protein expression levels in LSC-like cells from diagnostic AML patients independent of received therapy. L) Response scores of AML patients with number of structural variants. M) Response score of AML patients with recurrent AML mutations. Each dot represents an AML patient sample with the line marking the mean unless specified otherwise. Mann-Whitney test was used to compare groups and log-rank test to compare therapy durations of AML patients.

FIG. 5: A) IC₅₀s for VEN of 19 AML cell lines classified as primitive (Prim-AML, N=11) or monocytic (Mono-AML, N=8) treated ex vivo with 1.5 μM of 5-AZA and increasing concentrations of VEN for 72 hours. Representative data of two independent experiments. Each dot represents a cell line with the line marking the mean. Mann-Whitney test was used to compare the two groups. B) Pre-treatment clinical characteristics of 54 AML patients treated with 5-AZA/VEN as first-line therapy, including age, percentage of bone marrow blasts, peripheral leukocyte counts and percentage of CD34′ cells. Each dot represents an individual AML patient. C) Percentage of CD64′ AML cells of refractory AML patients pre-treatment and after induction at day 15 and day 30. Dashed lines connect different time points of the same patient.

FIG. 6: A) Percentages of Mature, Non-LSC-like and LSC-like populations among bulk AML cells in 72 diagnostic AML samples sorted by frequency of LSC population. B) Top 50 differentially expressed genes between Mature and LSC-like populations from specific to Prim- or Mono-AMLs or shared by both classes. C) Gene-set enrichment analysis (GSEA) results comparing LSC-like and Mature population from Mono-AMLs. NES: Normalized Enrichment Score. D) Transcriptome-based LSC scores in LSC-like and Mature populations. E) PCA plot of bulk RNA-seq data from LSC-like and Mature subpopulations from Prim-AMLs (N=11) and Mono-AMLs (N=6) annotated based on mutation and subpopulations. F) Heatmap of apoptosis regulators in LSC-like and Mature subpopulations from both AML classes. J) Mean Fluorescence Intensity (MFI) of BCL-xL in (left) AML bulk and (right) LSC-like and Mature subpopulations from Prim-AML (N=11) or Mono-AML (N=7) patient samples. Two-Way ANOVA with Tukey correction for multiple comparisons test was used to compared groups of four and Mann-Whitney test was used to compare groups of two unless specified otherwise. Each dot represents an AML patient sample with the line marking the mean unless specified otherwise.

FIG. 7: A) Schematic representation of experimental design for B-D. BCL-2 family protein expression levels and response to ex vivo 5-AZA/VEN treatment of MNCs from diagnostic AML patient samples was assessed by flow cytometry. B-E) Treatment-naïve AML patient samples were stratified based on ex vivo cell viability in LSC-like cells after 24 h and plotted for pre-culture BCL-2. MCL-1 or BCL-xL MFI z-scores or response score calculated based on normalized BCL-2 family protein expression levels in LSC-like cells from diagnostic AML patients. Each dot represents an AML patient sample with the line marking the mean unless specified otherwise. Mann-Whitney test was used to compare groups.

FIG. 8: A-D) BCL-2, MCL-1 or BCL-xL z-scores in LSC-like cells from diagnostic AML patients receiving first line HMA/VEN therapy with known outcomes. E) Duration of HMA/VEN therapy represented as therapy cycles of AML patients from both cohorts combined with above (>0.4) and below (<0.4) median response score. F) Duration of HMA/VEN therapy represented as therapy cycles of AML patients who achieve complete remission from both cohorts combined with above (>0.4) and below (<0.4) median response score. G-I) Response score was calculated based on normalized BCL-2 family protein expression levels in bulk AML cells from diagnostic AML patients receiving first line HMA/VEN therapy with known outcomes from cohort 1 and cohort 2. H) Response score was calculated based on normalized BCL-2 family protein expression levels in bulk AML cells from diagnostic AML patients receiving first line standard chemotherapy with daunorubicine/cytarabine (DA). I) Patient characteristics of 73 diagnostic AML samples with retrospectively assessed factors associated with response score. Univanant linear regression was performed for every parameter. Multivariant logistic was performed on parameters with p<0.1 in the univariant analysis.

FIG. 9: A) Schematic representation of experimental design for B-D. BCL-2 family protein expression levels and response to ex vivo 5-AZA/VEN treatment of mononuclear cells from diagnostic AML patient samples was assessed by flow cytometry. B-D) Treatment-naïve AML patient samples were stratified based on ex vivo cell viability in LSC-like cells after 24 h and plotted for pre-culture BCL-2, MCL-1 or BCL-xL Mean Fluorescence Intensity (MFI) z-scores or E) Response Score calculated based on normalized BCL-2 family protein expression levels in LSC-like cells from diagnostic AML patients. Each dot represents an AML patient sample with the line marking the mean unless specified otherwise. Mann-Whitney test was used to compare groups. LSC: Leukemic stem cell

FIG. 10: Response to 5-AZA/VEN therapy in AML patients can be predicted by Response-scoring in LSC-like cells
A) Schematic representation of experimental design for B-D. B-G) Mononuclear cells of AML patient samples treated first-line with 5-AZA/VEN from three independently processed cohorts (Cohort 1: n=17, Cohort 2: n=18, Cohort 3: n=24) were stained with surface antibodies, followed by intracellular staining of three BCL-2 family proteins. Response Score was calculated based on normalized BCL-2 family protein expression levels in LSC-like, non-LSC, Mature and total blast cells. B) Expression of BCL-2, MCL-1 and BCL-xL in LSC-like cells of AML patients from cohorts 1 and 2 combined and associated 5-AZA/VEN therapy outcome. Protein expression shown as Mean Fluorescence Intensity (MFI) z-scores. C) Response Score in LSC-like cells of AML patients from cohorts 1 and 2 combined and association to 5-AZA/VEN therapy outcome. D) Comparison of Response Score in LSC-like, non-LSC. Mature and total blast cells of AML patients from cohorts 1 and 2 and association to 5-AZA/VEN therapy outcome. E) Event-free survival of first-line 5-AZA/VEN AML patients from cohorts 1 and 2 combined with above and below median Response Score, BCL-2 expression, MCL-1 expression or BCL-xL expression in LSC-like cells. F) Response Score in LSC-like cells of AML patients from cohort 3 and associated 5-AZA/VEN therapy outcome. G) Event-free survival of first-line 5-AZA/VEN AML patients from cohort 3 with above (>0.4) and below (<0.4) median Response Score in LSC-like cells. H Schematic representation of experimental design for I-J. Mononuclear cells of relapsed/refractory AML patients who received 5-AZA/VEN as a salvage therapy (Cohort 4: n=23) were stained with surface antibodies, followed by intracellular staining of BCL-2 family proteins. 1) Response Score in LSC-like cells of AML patients from cohort 4 and associated 5-AZA/VEN therapy outcome. J) Event-free survival of salvage-treated 5-AZA/VEN AML patients from cohort 4 with above (>0.4) and below (<0.4) median Response Score determined in LSC-like cells. Each dot represents an AML patient sample with the line marking the mean unless specified otherwise. Mann-Whitney test was used to compare groups and log-rank test to compare therapy durations of AML patients. LSC: Leukemic stem cell, MFI: Mean Fluorescence Intensity, 5-AZA: Azacitidine, VEN: Venetoclax, R/R: Relapse/Refractory to standard induction.

FIG. 11: Response Score in LSC-like cells predicts response to 5-AZA/VEN with high accuracy.
A) Response Score in LSC-like cells of AML patients from all first-line 5-AZA/VEN AML patients combined (cohorts 1, 2 and 3) and association to 5-AZA/VEN therapy outcome. B) Receiver operating characteristic (ROC) curve of Response Score and therapy outcomes of all first-line 5-AZA/VEN AML patients combined (cohorts 1, 2 and 3). C) Event-free survival of all first-line 5-AZA/VEN AML patients combined (cohorts 1, 2 and 3) with above and below median Response Score. D) Event-free survival of all first-line 5-AZA/VEN AML patients who achieved complete remission from combined cohorts (cohorts 1, 2 and 3) with above (>0.4) and below (<0.4) median Response Score. E) Patient characteristics of first-line 5-AZA/VEN cohorts with retrospectively assessed risk factors of refractoriness to therapy. Univariate logistic regression was performed for every parameter. Multivariate logistic regression was performed on parameters with p<0.15 in the univariate analysis. F) Event-free survival from combined cohorts (cohorts 1, 2 and 3) with above (>0.4) and below (<0.4) median Response Score based in patients with complex karyotype, RUNX1 or NPM1 mutation. G) Response Score in LSC-like cells from diagnostic AML patients receiving first-line standard induction chemotherapy and association to therapy outcome. H) Schematic representation of experimental design for I-J. Response Score was calculated based on normalized BCL-2 family protein expression levels in LSC-like cells from diagnostic AML patients independent of received therapy (n=95). I) Response Score of AML patients differentiated by the number of structural variants. J) Response Score of AML patients with different recurrent AML mutations. K) Schematic model outlining the Response Score concept for predicting clinical response to 5-AZA/VEN. Each dot represents an AML patient sample with the line marking the mean unless specified otherwise. Mann-Whitney test was used to compare groups and log-rank test to compare therapy durations of AML patients. LSC: Leukemic stem cell, 5-AZA: Azacitidine, VEN: Venetoclax, ROC: Receiver operating characteristic, ORR: objective response rate, Resp: Responder.

FIG. 12: A-M) Comparison of Response Score and BH3-profilin in a selected subset of 5-AZA/VEN first-line in AML patient samples from cohorts 1, 2 and 3 (N=15). A) Response Score in LSC-like cells with 5-AZA/VEN therapy outcome. B-C) Event-free survival of first-line 5-AZA/VEN patients stratified by above and below B) Median Response Scores of selected samples or C) previously defined 0.4 cut-off. D-G) BH3-profiling results plotted as 5-AZA/VEN therapy outcome with AUC of cytochrome C release in LSC-like cells mediated by D) VEN, E) HRK, F) MS1 and G) combination of HRK and MS1. H) Event-free survival of first-line 5-AZA/VEN patients stratified by above and below median of VEN AUC in LSC-like cells. I-L) BH3-profiling results plotted as 5-AZA/VEN therapy outcome with AUC of cytochrome C release in total blast cells mediated by I) VEN, J) HRK, K) MS1 and L) combination of HRK and MS1. M) Event-free survival of first-line 5-AZA/VEN AML patients stratified by above and below median of VEN AUC in total blast cells. Each dot represents an AML patient sample with the line marking the mean unless specified otherwise. Mann-Whitney test was used to compare groups. LSC: Leukemic stem cell, 5-AZA: Azacitidine, VEN: Venetoclax.

### EXAMPLES

The Examples shall merely illustrate the invention. They shall, whatsoever, not be construed as limiting the scope.

### Example 1: General Methods

### Primary AML Patient Samples

AML samples were collected from diagnostic peripheral blood (PB) aspirations at the University hospital in Heidelberg in accordance with the Declaration of Helsinki after obtaining written consent from each patient. The project was approved by the Ethics Committee of the Medical Faculty of Heidelberg (S-169/217, S-648-2021). PB mononuclear cells (MNCs) were isolated by density gradient centrifugation using Ficoll Paque Plus (GE Healthcare, cat #GE17-1440-03), and stored in liquid nitrogen until further use. An experienced hematologist categorized all patients into responders and non-responders by retrospectively reviewing all pathology and flow cytometry reports. Responders were defined as those who achieved blast reduction to <5% in accordance with ELN.

### Processing of Primary AML Cells

Viably cryopreserved AML PB samples were thawed at 37° C. in Iscove's modified Dulbecco's medium (IMDM) containing 10% FBS, and treated with DNase I for 15 min (100 μg/ml).

### Ex Vivo Drug Screening in Primary Leukemia Cells

Recovered cells were cultured using previously established protocols using IMDM, 15% BIT (bovine serum albumin, insulin, transferrin; Stem Cell Technologies, cat #09500), 100 ng/ml SCF (PeproTech, cat #300-07), 50 ng/ml FLT3-L (PeproTech, cat #300-19), 20 ng/ml IL-3 (PeproTech, cat #200-03), 20 ng/ml G-CSF (PeproTech, cat #300-23), 100 μM β-mercaptoethanol (ThermoFisher, cat #31350010), 500 nM SRI (StemRegenin 1, STEMCELL Technologies, cat #72342), 1 μM UM729 (STEMCELL Technologies, cat #72332), and 1% penicillin-streptomycin (Sigma, cat #P4458-100 ML). For drug assay in FIG. 1, 0.5×10⁵ AML cells/well were seeded in flat-bottom %-well plates, and cells were treated with increasing concentration of Azacitidine (0.5 μM, 1.5 μM, 4.5 μM, 13.5 μM, 40.5 μM) and Venetoclax (0.3 nM, 0.9 nM, 2.7 nM, 8.1 nM, 24.3 nM, 72.9 nM, 218.7 nM, 656.1 nM, 1968.3 nM) alone and in combination for 72 h. After 72 h, viability was assessed using CellTiter-Glo (Promega, cat #G7571) Luminescent Cell Viability Assay. For drug assay in FIG. 3, 0.5×10⁵ AML cells/well were seeded in flat-bottom 96-well plates, and cells were treated with 1.5 μM of Azacitidine and 100 nM of Venetoclax for 24 h. After 24 h, the cells were stained with a total of 11 fluorescent cell surface antibodies. Same amount of CountBright Absolute Counting Beads (Thermo Fisher Scientific, cat #C36950) together with 7-AAD (BD Biosciences, cat #559925) were added to each sample prior to analysis with BD LSRFortessa Cell Analyzer.

### Intracellular Staining for BCL-2 Family Members

Thawed cells were stained with Zombie NIR Fixable Viability stain in PBS (BioLegend, cat #423105), followed by staining with a total of 11 fluorescent cell surface antibodies. Stained cells were fixed and permeabilized using the Fixation/Permeabilization Solution Kit (BD Biosciences, cat #554714) according to manufacturer's instructions, followed by a secondary permeabilization step for enhanced intracellular staining using Permeabilization Buffer Plus (BD Biosciences, cat #561651). Fixed and permeabilized cells were stained separately for anti-human-BCL-2-PE (clone 124, Cell Signaling, cat #26295S), anti-human-MCL-1-PE (clone D2W9E, Cell Signaling, cat #65617S) and anti-human-MCL-1-PE (clone 54H6, Cell Signaling, cat #13835S). Samples were analyzed with BD LSRFortessa Cell Analyzer.

### BH3 Profiling of Primary AML Samples

Thawed cells were stained with Zombie NIR Fixable Viability stain in PBS (BioLegend, cat #423105), followed by staining with a total of nine fluorescent cell surface antibodies. BH3 profiling was performed as previously described. Cells were exposed to increasing concentrations of synthetic BH3 peptides as well as BH mimetics in MEB buffer (150 mM mannitol, 10 mM HEPES-KOH pH 7.5, 50 mM KCl, 0.02 mM EGTA, 0.02 mM EDTA, 0.1% BSA and 5 mM Succinate) for 60 minutes after plasma membrane permeabilization with digitonin (0.002%). After 60 minutes peptide/mimetic exposure at room temperature cells were fixed using 4% formaldehyde for 15 minutes, followed by neutralization for 10 minutes using N2 buffer (1.7M Tris, 1.25M Glycine pH 9.1). Sensitivity to BH3 peptides/mimetics were measured as Cytochrome C release using anti-Cytochrome C FITC antibody (clone 6H2.B4, Biolegend, cat #612302) via BD FACSymphony A3 Cell Analyzer. DMSO was used as a negative control for Cytochrome C retention, whereas Alamethicin (ALM) was used as a positive control for 100% Cytochrome C release. Cytochrome C loss was calculated using the following equation: [Cytochrome C release=1− [symbol] MFI [symbol] _sample [symbol] MFI [symbol] _ALM)/ [symbol] MFI [symbol] _DMSO]. Cytochrome C release was assessed in each FACS-gated population. To assess the variation of Cytochrome C release as a function of BH3 peptide/mimetic concentration, area under the curve (AUC) for each BH3 peptide/mimetic was calculated using Prism v. 9.

### Longitudinally Collected Primary AML Samples

From three de novo AML patients (AML55, AML61 and AML62). PB was drawn prior to treatment with 5-AZA/Ven (Day 0), followed by blood draws during therapy (Day 1-6). PB-MNCs were isolated as explained above. Prior to freezing, 0.2×10⁶ cells were stained with a total of 11 fluorescent cell surface antibodies and analyzed with BD LSRFortessa Cell Analyzer.

### Processing of AML Cell Lines

Twenty-four AML cell lines were cultivated at 37° C. in a humidified incubator with 5% CO2. All cell lines were authenticated.

### In Vitro Drug Screening in Leukemia Cell Lines

0.1×10⁵ cells/well from each cell line were seeded in flat-bottom 96-well plates, and cells were treated with increasing concentration of Venetoclax (1 nM, 3 nM, 9 nM, 27 nM, 81 nM, 243 nM, 729 nM, 2187 nM) in combination with a single dose of Azacitidine (1.5 μM) for 72 h. After 72 h, viability was assessed using CellTiter-Glo (Promega. cat #G7571) Luminescent Cell Viability Assay.

### Fluorescent Activated Cell Sorting (FACS)

Primary AML cells were stained with a total of ten fluorescent cell surface antibodies. Cells were sorted into four populations according to CD11b, CD64 and GPR56 expression within the lineage-negative gate. Cells from each population and from lineage-negative bulk were sorted directly into RNA extraction buffer (Thermo Fisher, cat #KIT0214), snap-frozen and stored at −80° C. until RNA extraction. For xenotransplantations, cells from each population were sorted into PBS.

### Determination of In Vivo Leukemia-Initiating Potential

NOD.Prkdcscid.Il2rgnull (NSG) mice were bred and housed under specific pathogen-free conditions at the central animal facility of the German Cancer Research Center (DKFZ). Animal experiments were conducted in compliance with all relevant ethical regulations. All experiments were approved by the Regierungsprdsidium Karlsruhe under Tierversuchsantrag numbers G-140-21 and G42/18.

### Xenotransplantations

Female mice 8-12 weeks of age were sublethally irradiated (175 cGy) 24 h before xenotransplantation assays. Up to 1×10⁶ cells from FACS sorted primary AML samples (see above) were injected into the femoral BM cavity of sublethally irradiated mice. Human leukemic engraftment in mouse BM was evaluated by flow cytometry (maximum 45 weeks unless end point criteria were reached earlier) using anti-human-CD45-FITC (clone H130), anti-human-CD34-BUV395 (clone 581), anti-human-GPR56-PE (clone CG4), anti-human-CD19-FITC (clone HIB19), anti-human-CD33-PE-Cy7 (clone WM53), CD64-APC, CD11b-BV711 and anti-mouse-CD45-Alexa700 (clone 30-F11). At end point. BM cells were harvested from tibiae, femurs, and iliac crests by bone crushing. Spleen cells were harvested by mincing the spleen with a plunger. Following red blood cell lysis, cells were resuspended in FBS+10% DMSO (Sigma, cat #D2650-100) and stored in liquid nitrogen until further use.

### RNA Sequencing AML Populations

RNA extraction and purification of FACS-sorted cells was done using PicoPure RNA Isolation Kit according to manufacturer's instructions (Thermo Fisher, cat #KIT0214). RNA quality assessment and quantification were performed with Bioanalyzer using Agilent RNA 6000 Pico Kit (Agilent, cat #5067-1513). Whole transcriptome amplification was performed using a modified smart-seq2 protocol, with 5 pd of a modified RT buffer containing 1′ SMART First Strand Buffer (Takara Bio Clontech, cat #639538), 1 mM dithiothreitol (Takara Bio Clontech), 1 μM template switching oligo (IDT), 10 U pd-1 SMARTScribe (Takara Bio Clontech, cat #639538) and 1 U μl-1 RNasin Plus RNase Inhibitor (Promega, cat #N2615). Tagmentation of cDNA was done using Nextera XT DNA Library Preparation Kit (Illumina, cat #FC-121-1030). All RNA libraries were pooled and sequenced together on an Illumina NextSeq 550 high output sequencer (1.4 μM with 1% PhiX loading concentration, single-end 75 bp read configuration).

### Raw Data Processing and Quality Control of RNA Sequencing Data

Reads were demultiplexed, and STAR aligner v. 2.5.3a was used to align FASTQ files containing reads for individual samples by two-pass alignment. Reads were aligned to a STAR index generated from the 1000 Genomes Project human genomes assembly (hs37d5), using GENCODE v.19 gene models. Default alignment call parameters were used with the following modifications. --outSAMtype BAM Unsorted SortedByCoordinate--limitBAMsortRAM 100000000000--outBAMsortingThreadN=1-outSAMstrandField intronMotif--outSAMunmapped Within KeepPairs--outFilterMultimapNmax 1--outFilterMismatchNmax 5--outFilterMismatchNoverLmax 0.3--chimSegmentMin 15--chimScoreMin 1-chimScoreJunctionNonGTAG 0-chimJunctionOverhangMin 15--chimSegmentReadGapMax 3-alignSJstitchMismatchNmax 5-1 5 5--alignIntronMax 1100000--alignMatesGapMax 1100000--alignSJDBoverhangMin 3--alignIntronMin 20.

Sambamba v.0.6.5 was used for the alignment file sorting, duplicate marking and BAM index generation using eight threads. Quality control analysis was performed using the sambamba flagstat command and maseqc v.1.1.8 with the hs37d5 assembly and GENCODE v.19 gene models. Depth of Coverage analysis for maseqc was turned off. Gene-specific gene counting over exon features based on GENCODE v.19 gene models was performed using featureCounts v.1.5.1. Quality threshold was set to 255, which indicates that STAR found a unique alignment. Strand-unspecific counting was used.

DESeq2 was used for statistical analysis of the read counts to identify differentially-expressed genes between the LSC and Mature populations in Prim-AMLs and Mono-AMLs. Genes with an FDR-corrected p-value ≤0.05 and at least a 1.5-log fold change in expression (|log2FC|≥1.5) were considered as differentially expressed. Gene expression estimates for PCA visualization were adjusted by variance stabilization. Gene set enrichment analysis for Hallmark gene sets between LSCs and Mature cells was performed based on log fold change order-ranked gene list. LSC17 scores were calculated for each AML sample as the mean expression of the normalized log 2-transformed gene counts of the 17 LSC signature genes from Ng et al as follows:

$$LSC 17 score = 1 / 17 \times ( DNMT 3 B + ZBTB 46 + NYNRIN + ARHGAP 22 + LAPTM 4 B + MMRN 1 + DPYSL 3 + KIAA 0125 + CDK 6 + CPXM 1 + SOCS 2 + SMIM 24 + EMP 1 + NGFRAP 1 + CD 34 + AKR 1 C 3 + GPR 56 )$$

### Quantification and Statistical Analysis

Flow cytometry data analysis was done using FlowJo v.10.5.3. TSNE plots for BCL-2 family members (FIG. 3) and 5-AZA/VEN sensitivities (FIG. 4) were done using the FlowJo TSne plugin v.2.0.0. All statistical analyses excluding RNA sequencing data were done using Prism v.9.

### Example 2: Ex Vivo Resistance of AML to 5-AZA/VEN is Associated with Monocytic Differentiation

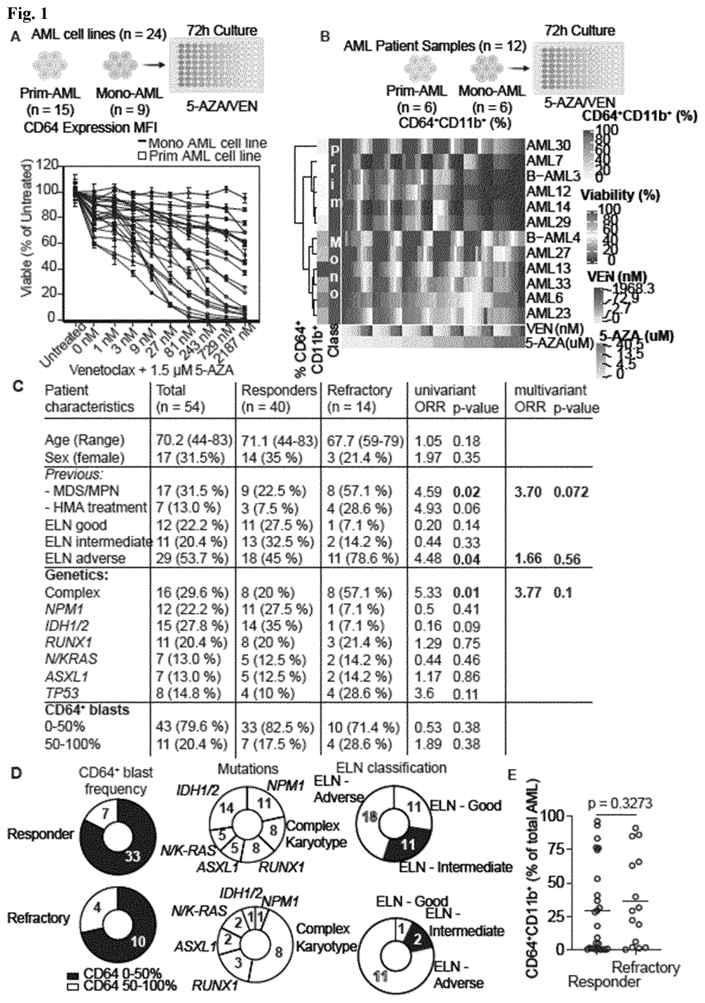

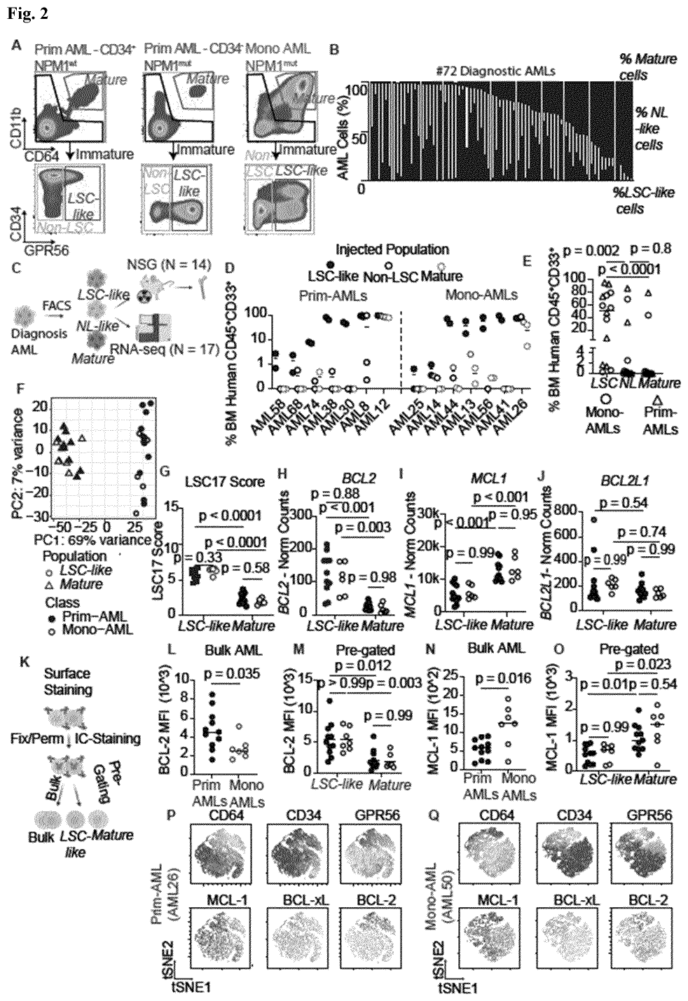

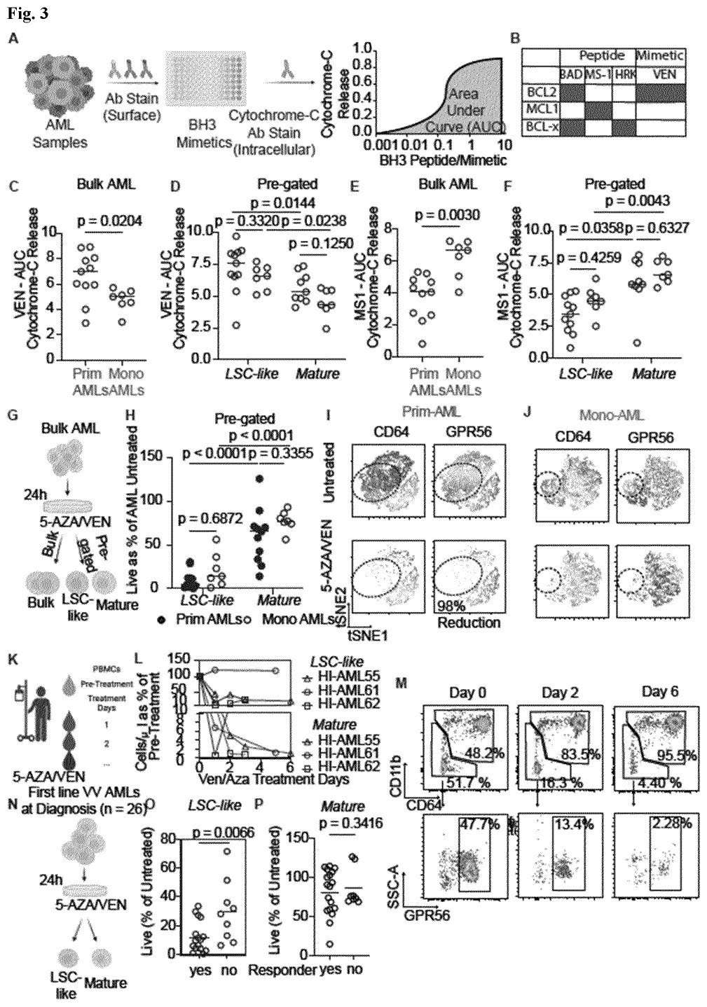

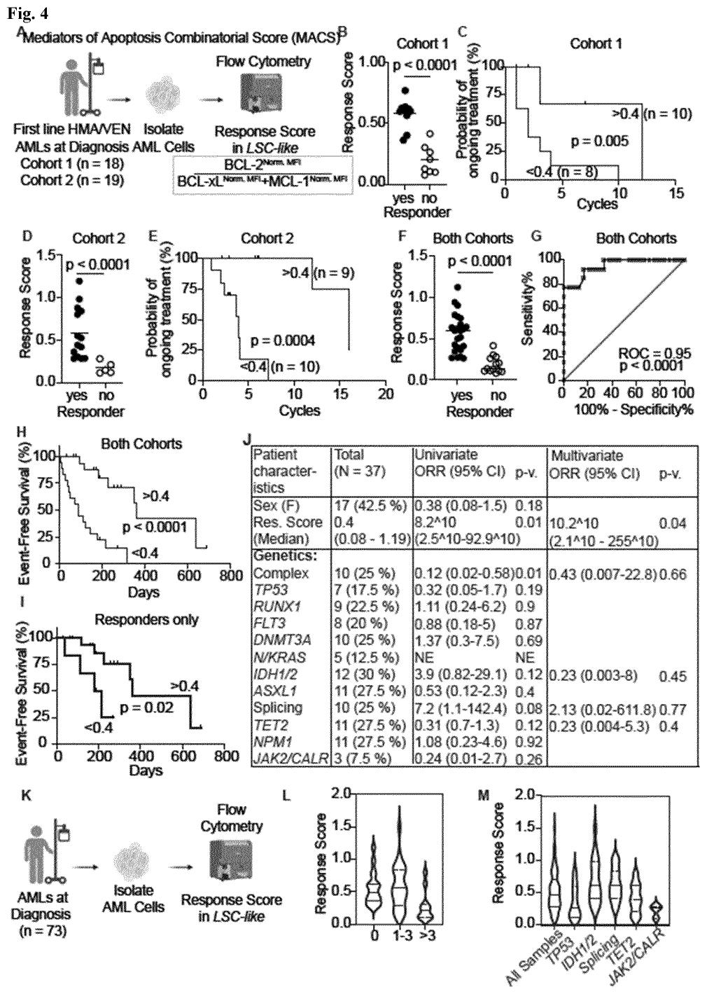

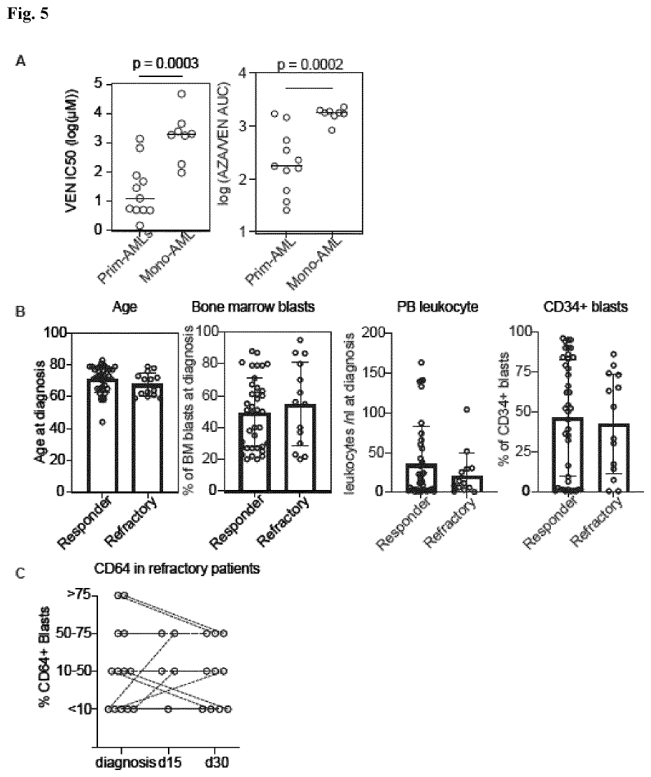

To identify predictive parameters for the response to 5-AZA/VEN treatment, the in vitro sensitivity of 19 AML cell lines treated for 72 h was evaluated. After stratifying these based on the mean fluorescent intensity (MFI) of the monocytic marker CD64 into monocyte-like AMLs (Mono-AML) and primitive-like AMLs (Prim-AML), it was observed that Mono-AML cell lines were highly resistant to 5-AZA/VEN while the majority of Prim-AML cell lines were sensitive even at low concentrations of VEN (FIG. 1A). Overall, the mean IC₅₀ of Mono-AML cell lines was 155-fold higher compared to Prim-AML cell lines (1901 nM vs 12 nM) (FIG. 5A). To validate the 5-AZA/VEN resistance of Mono-AMLs observed in cell lines, primary cells from 12 AML patients were treated with increasing concentrations of 5-AZA and VEN (FIG. 1B). Unbiased clustering on the cell viability measured after 72 h of ex vivo treatment revealed two independent clusters. The cluster associated with treatment resistance contained exclusively samples with >40% CD64⁺CD11b⁺ cells pre-treatment (FIG. 1B, bottom cluster), while specimens <20% CD64⁺CD11b⁺ cells were highly sensitive to VEN-based treatment and clustered separately (FIG. 1B, top clusters). This data implies, that monocytic differentiation of bulk AML cells is associated with ex vivo resistance to 5-AZA/VEN treatment. The CD64⁺CD11b⁺ percentages obtained from the unbiased clustering were used onwards to stratify AML patient samples into Mono-AML (>40%) and Prim-AML (<20%).

### Example 3: Clinical Response of AML to 5-AZA/VEN is Independent of Monocytic Differentiation

To determine the significance of the aforementioned findings in the clinical context, a cohort of 54 previously untreated patients with newly diagnosed AML who received HMA/VEN (e.g. 5-AZA/VEN) at Heidelberg University Hospital between 2019 and 2022 for parameters associated with refractoriness to the treatment was analyzed. In contrast to our ex vivo results, the frequency of CD64⁺ blast was not a significant predictor of refractoriness (FIG. 1C-E). In univariant logistic regression analysis, (1) previous Myelodysplastic Syndrome or Myeloproliferative Neoplasm (MDS/MPN), (2) adverse risk according to the 2017 ELN classification and (3) complex karyotype (CK) as the only significant factors predicting the risk of refractory disease (FIG. 1C-D) were identified. CK and MDS/MPN also trended towards independent predictors in the multivariant analysis, with 50% of these patients being refractory. ELN “adverse” was not reproduced as an independent predictor due to the high response rates of RUNX1 and ASXL1 mutated AMLs (FIG. 1C-D). This finding was substantiated by quantifying the percentage of CD64⁺ CD11b⁺ AML cells from cryostored samples of 41 first-line treated HMA/VEN patients, and an equal distribution of CD64⁺CD11b⁺ cells between responding and refractory patients (FIG. 1E) was confirmed. Lastly, to investigate outgrowth of monocytic clones, longitudinal flow cytometry reports available for 13 patients with primary refractory disease was studied. CD64 surface expression in bone marrow (BM) AML blasts on day 15 and/or day 30 post-treatment was not associated with therapy resistance (FIG. 5C). These data conflict with the ex vivo data reported above and by others and argues that monocytic differentiation is not a dominant predictor of response to HMA/VEN (e.g. 5-AZA/VEN).

### Example 4: LSCs are Enriched in Immature, GPR56⁺ Blasts in Monocytic and Primitive AML

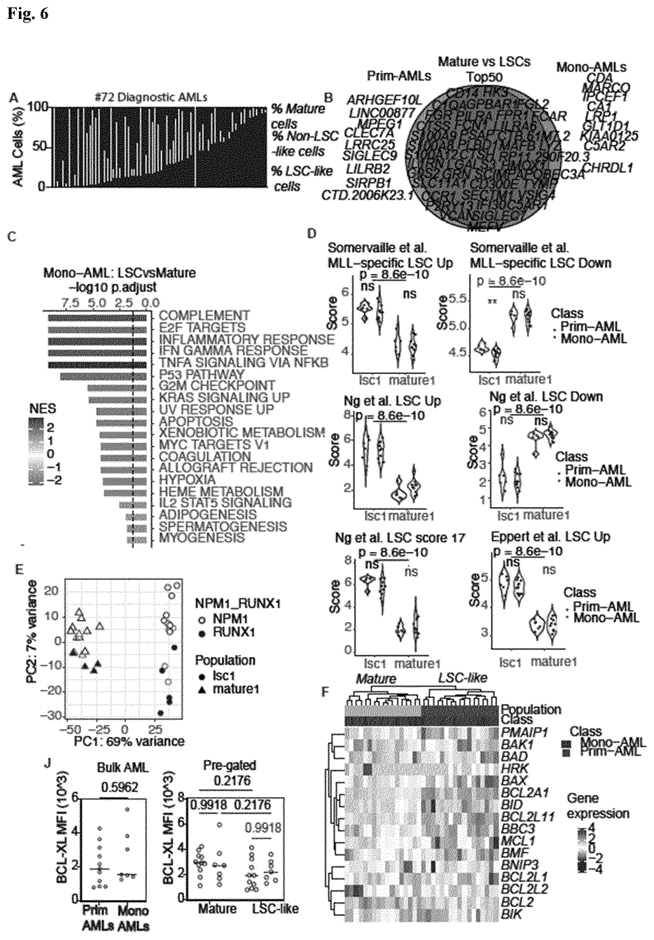

The discrepancy between clinical and pre-clinical data suggests that the analysis of bulk AML patient samples is not suitable to predict response to HMA/VEN treatment (e.g. 5-AZA/VEN treatment). Instead, it was hypothesized that LSCs drive resistance and relapse after HMA/VEN treatment and would therefore be the appropriate population for further exploration. However, as AML is a highly heterogeneous disease, reliable detection of LSCs across genetic subclasses has proven difficult. Therefore, the aim was to functionally and transcriptionally characterize LSCs in Mono-AML and Prim-AML samples. Cell surface expression of CD64 and CD11b readily distinguished two predominant cell populations in a cohort of 72 diagnostic AML samples: Mature CD64⁺CD11b⁺ cells (Mature) were the predominant population in Mono-AMLs, but were present in Prim-AMLs as well, although at much lower frequencies (40-97.6% vs. 0.1-20% of leukemic blasts) (FIG. 2A-B, FIG. 6A). To study subpopulations within Immature blasts, GPR56 expression, a marker for LSCs and adverse outcome in AML, was included to further enrich for functional LSCs in both Mono- and Prim-AML samples. A GPR56′ population ranging from 0.4 to 92.6% of all AML cells in all examined samples was identified, including Prim-AMLs and Mono-AMLs as well as NPM1 wild-type and mutated patient samples for which classical LSC surface markers, such as CD34, often fail to define leukemia initiating cells (FIG. 2A-B). To validate the association between GPR56 expression and leukemogenicity in Mono- and Prim-AML, FACS-sorted Mature, Immature GPR56⁻ (Non-LSCs) and Immature GPR56⁺ (LSC-like) populations from 12 patient samples were injected into NSG mice and assessed for leukemogenic potential (FIG. 2C). In 12/12 AMLs leukemic engrafiment (CD45⁺CD33⁺) was initiated by the LSC-like population while in only 2/12 patients Non-LSC-like and Mature fractions consistently generated relevant leukemic engraftment (FIG. 2D-E). Importantly, no differences in the leukemogenic potential of LSC-like cells derived from Mono-AMLs and Prim-AMLs were detected as both AML classes showed superior engraftment of the LSC-like population.

### Example 5: The Immature GPRS6⁺ Fraction is Enriched for Stemness-Associated Molecular Programs

Due to the similarities in the leukemogenic potential observed for Mono-AMLs and Prim-AMLs, LSC-like and mature cells were further characterized by performing RNA sequencing on sorted cell populations from 17 AML patients. Intriguingly, dimensionality reduction using principal component analysis (PCA) revealed clear clustering of the samples based on population (LSC-like and mature) but not based on the two AML classes (FIG. 2F). Differential gene expression analysis between LSC-like and Mature cells showed that the upregulated genes in LSC-like cells contained known cancer stem cell markers including KIT, ERG, GPR56 and PROM), whilst upregulated genes in Mature cells included monocytic markers such as S100A9, S100A8 and CD14 and were enriched for pathways associated with myeloid differentiation (FIG. 6B-C). Moreover, several stemness scores, including the LSC17 score, were significantly higher in LSC-like compared to Mature cells, irrespective of the AML class (FIG. 2G). As no striking differences between LSC-like cells from AML samples stratified based on their monocytic-like surface expression were observed, it was also assessed whether genetics would explain the subtle differences observed between different LSC-like samples in the PCA. Indeed, RUNX1-mutated LSC-like cells and NPM1-mutated LSC-like cells clustered separately (FIG. 6E). These findings highlight the transcriptomic stemness characteristics of functionally defined LSC-like cells and show that on the LSC-level the transcriptomic clustering is largely determined by the underlying mutational events instead of their differentiation state (e.g. of their blast progeny).

### Example 6: LSC-Like Cells Predominantly Express BCL-2 while MCL-1 is Highly Expressed in Mature Blasts

Intrigued by the indistinguishable clinical responses of Mono-AMLs and Prim-AMLs to HMA/VEN treatment (e.g. 5-AZA/VEN treatment), the expression of BCL-2 family members, such as BCL-2. MCL-1 and BCL-2L1, within the LSC-like subpopulation of either AML class was correlated. In line with previous studies, BCL-2 was highly expressed (4.7-fold higher) in the LSC-like populations compared to the Mature populations (FDR<0.01, FIG. 2I, whereas MCL-1 expression was higher (2.3-fold higher) in the Mature populations compared to LSC-like cells (FDR<0.01, FIG. 21). When comparing the expression of BCL-2 and MCL-1 within LSC-like and Mature cells, no differences between Mono-AMLs and Prim-AMLs samples were found. In contrast, BCL-2L1 expression, encoding for BCL-xL, was not skewed towards a particular subpopulation (FIG. 2J). Furthermore, transcriptomic analysis of the extended BCL-2-family also did not cluster based on AML class (FIG. 6F). These results suggest that LSC-like cells express higher levels of BCL-2 and lower levels of MCL-1 than more mature cells, irrespective of overall AML differentiation status. To confirm expression levels of the BCL-2 family members on the protein level, an intracellular staining protocol combining our surface marker panel with antibodies specific for BCL-2, MCL-1 and BCL-xL (FIG. 2K) was established. Without gating on LSC-like or Mature subpopulations, bulk AML cells of Prim-AMLs, showed a 1.8-fold higher expression of BCL-2 proteins compared to Mono-AMLs (FIG. 2L). The analysis of pre-gated subpopulations, however, showed that BCL-2 was highly expressed in LSC-like cells but not in Mature cells irrespective of AML class, abrogating the AML class driven differences observed in bulk (2.6-fold higher in LSC-like compared to Mature; FIG. 2M). Vice versa, assessment of MCL-1 protein in bulk revealed 2.1-fold higher levels in Mono-AML than Prim-AMLs (FIG. 2N). Again, after gating on the LSC-like and mature subpopulations, MCL-1 protein expression did not differ between Mono-AML and Prim-AMLs samples, with MCL-1 being highly expressed in the Mature population of both AML classes (2.1-fold higher compared to LSC-like cells; FIG. 2O). Meanwhile, BCL-xL showed no clear subpopulation nor AML class specific differences (FIG. 6J). Representative t-distributed stochastic neighbor embedding (tSNE) plots of two cases demonstrate that Prim-AMLs contain more immature LSC-like cells, expressing higher levels of CD34 and GPR56 together with BCL-2, but also contain a smaller fraction of Mature cells expressing CD64 and MCL-1 (FIG. 2P). On the contrary. Mono-AMLs are enriched for more Mature cells expressing high levels of CD64 and MCL-1, and contain only a small immature LSC-like population expressing CD34, GPR56 and BCL-2 (FIG. 2Q). Taken together, assessment of BCL-2, MCL-1 and BCL-xL protein levels supports the findings of the transcriptional data, and highlights the similarities of the subpopulations of Prim-AMLs and Mono-AMLs despite the fact that their frequencies vary extensively in the two different AML classes.

### Example 7: BH3-Profiling Confirms that LSC-Like Cells of Both AML Classes are BCL-2 Dependent

Next, it was studied whether the subpopulation-specific expression differences of BCL-2 family members translate into functional consequences. To address this, BH3 profiling was performed to measure activity of apoptotic pathways in the same AML samples. Similar to the intracellular staining of BCL-2 family members, the surface markers were combined with BH3 profiling to assess mitochondrial apoptotic priming and dependence on pro-survival BCL-2 family proteins in bulk AML as well as in pre-gated LSC-like and Mature cells. Briefly, cells were exposed to pro-apoptotic BH3 peptides plus different mimetics to assess the release of mitochondrial cytochrome C, irreversibly committing a cell to undergoing apoptosis (FIG. 3A-B). In bulk AML cells, dependence on BCL-2 was higher in Prim-AMLs compared to Mono-AMLs upon exposure to BH3 mimetic VEN (FIG. 3C). However, differential analysis on pre-gated LSC-like and Mature cells abrogated this difference while revealing that LSC-like cells were more dependent on BCL-2 compared to the Mature cells in both AML classes (FIG. 3D). The exact opposite behaviour was found after assessing the dependency on MCL-1 via cytochrome C release upon exposure to the BH3 peptide MS-1 (FIG. 3E-F). Here Mature cells showed a 1.7-fold higher MS-1 mediated apoptotic priming compared to LSC-like cells, irrespective of the AML class. Taken together, this data suggests that the differences in the mRNA and protein expression of BCL-2 and MCL-1 predict subpopulation (LSC-like/Mature) specific dependencies, which are independent of AML class. In general, LSC-like cells were most dependent on BCL-2.

### Example 8: Ex Vivo 5-AZA/VEN Treatment Eradicates LSC-Like and Spares Mature Blasts in Both AML Classes

Since BH3-profiling indicated that LSC-like cells depend on BCL-2 and Mature cells on MCL-1, it was hypothesized that differential ex vivo treatment response of bulk Prim/Mono-AMLs is mainly driven by these subpopulations. Therefore, bulk AML cells from 16 newly diagnosed patients were exposed to 5-AZA/VEN for 24 h and the viability of bulk cells and subpopulations in comparison to untreated controls was analysed by flow cytometry (FIG. 3G). As found in FIG. 1B, bulk cells from Mono-AML patient samples exhibited a significantly higher resistance to the treatment (compared to Prim-AMLs). The analysis of subpopulation viability post-5-AZA/VEN revealed that in both AML classes 5-AZA/VEN only marginally reduced the Mature population by 40+/−32% (Prim-AML) and 22.5+/−11.5% (Mono-AML) (FIG. 3H). In comparison, LSC-like cells derived from both Prim-AML and Mono-AML classes were efficiently eliminated at 90+/−10.6% and 79+/−19%, respectively (FIG. 3H). Representative tSNE plots of viable cells from two AML samples were overlaid with heatmaps of CD64 and GPR56 expression to identify Mature and LSC-like fractions, respectively (FIG. 21-J). The differential response between Mature and LSC-like cells was further corroborated in the remaining viable cells as LSC-like cells were replaced by an enrichment of Mature cells independent of AML classes (FIG. 21-J). In line with the results from BH3-profiling and of BCL-2 expression, these data show that the differences in bulk AML sensitivity are mainly driven by the initial difference in proportions of the treatment-resistant but not disease propagating Mature subpopulation between Prim-AML and Mono-AML samples. Importantly, LSC-like cells numbers were effectively eliminated by 5-AZA/VEN in both AML classes.

### Example 9: HMA/VEN Rapidly Clears LSC-Ike Cells in Responsive Patients

Ex vivo treatment of frozen AML samples is notoriously challenging and hence prone to misinterpretations. Therefore, it was aimed to confirm the potential of HMA/VEN (e.g. 5-AZA/VEN) to eradicate LSCs-like cells in patients. For this purpose. PBMCs were collected from 3 AML patients with peripheral blasts before treatment initiation (Day 0) and during HMA/VEN (e.g. 5-AZA/VEN) therapy between Days 1-6 (FIG. 3K) and Mature and LSC-like populations size was normalized at each timepoint to pre-treatment cell counts. All three patients responded to therapy and achieved a significant reduction of the peripheral blast count within the first 24 hours of treatment. While the relative number of LSC-like cells decreased and remained low, a significant fraction of Mature cells persisted in all three patients during the first days of treatment (FIG. 3L-M). Lastly, ability of ex vivo 5-AZA/VEN treatment to predict clinical response was assessed. Therefore, viability of LSC-like and Mature fractions was compared after 24 h ex vivo 5-AZA/VEN treatment in 26 patients with known clinical response to first-line HMA/VEN (FIG. 3N). LSC-like cells from refractory patients were significantly more resistant to treatment while mature cells remained largely unaffected by the ex vivo treatment independent of the patient's clinical response (FIG. 3O-P). This data highlights the need to study disease driving subpopulations in AML and demonstrates functional properties of LSC-like cells as predictive for response to HMA/VEN treatment (e.g. 5-AZA/VEN).

### Example 10: Rapid and Robust Prediction of Ex Vivo Response with BCL-2 Family-Based LSC-Like Response Score

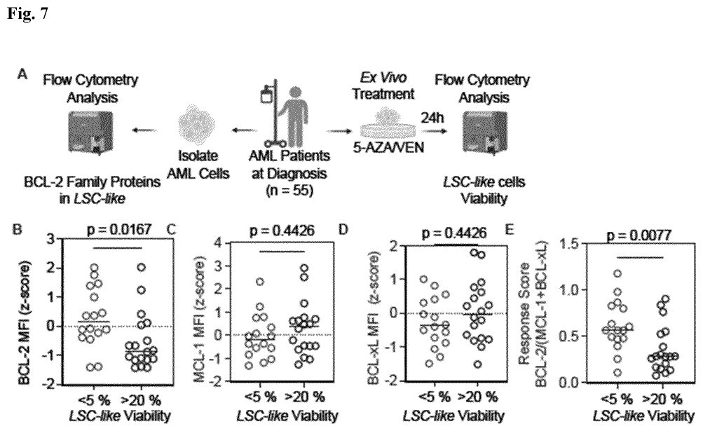

Flow cytometry is a routine diagnostic tool for the diagnosis and monitoring of AML in the clinic and can support clinical decision-making within several hours after sampling. Encouraged by the observed BCL-2 dependency in LSC-like cells and correlation of their in vitro viability with clinical response, it was hypothesized that BCL-2 family protein expression levels in LSC-like cells could predict patient response to HMA/VEN (e.g. 5-AZA/VEN) treatment at diagnosis. Ex vivo 5-AZA/VEN treatment and simultaneously intracellular staining of BCL-2. MCL-1 and BCL-xL on 55 diagnostic AML patient samples (FIG. 7A) were performed. Stratification based on the viability of LSC-like cells (<5% or >20%) after 24 h ex vivo 5-AZA/VEN treatment showed significantly higher intracellular BCL-2 expression scores in AML specimens sensitive to ex vivo treatment (FIG. 7B). While BCL-2 conveys sensitivity, MCL-1 and BCL-xL can inhibit apoptosis independent of BCL-2. Thus, all three proteins were incorporated into a singular response score of normalized expression of drug target to normalized expression of resistance factors: BCL-2^{Norm MFI}/(MCL-1^{Norm.MFI}+BCL-xL^{Norm.MFI}) Including alternative inhibitors of apoptosis and therefore potentially mediators of VEN resistance further improved separation between 5-AZA/VEN ex vivo responders and non-responders, highlighting the benefit of combinatorial assessment of BCL-2 family proteins within LSC-like cells. (FIG. 7C-E).

### Example 11: Rapid and Robust Prediction of Clinical Response and Remission Duration with LSC-Like Response Score

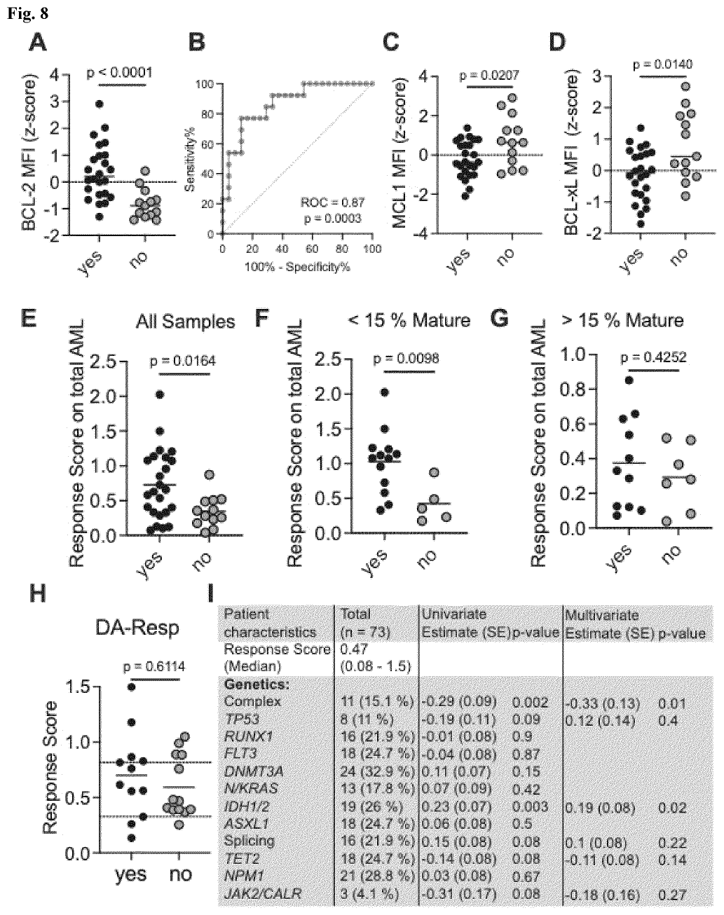

Encouraged by the aforementioned ex vivo results, it was assessed whether the response score in LSC-like cells could also be used to predict clinical response to HMA/VEN treatment. Expression of BCL-2 family proteins BCL-2, MCL-1 and BCL-xL was analyzed together with surface staining in diagnostic samples. Here, BCL-2 was higher expressed in LSC-like cells of patients achieving CR, CRi or MLFS, while MCL-1 and Bcl-xL were higher in patients with SD, PR or PD. However, not a single BCL-2-family protein alone gave clear separation of responders or non-responders, as levels of all 3 were highly variable (FIG. 8A-D). Therefore, the predictive value not of individual BCL-2 family proteins but of the combinatorial response score in LSC-like cells of two independent patient cohorts receiving HMA/VEN as first-line treatment (FIG. 4A) was assessed. Cohort 1 (n=18) showed significantly higher response scores in LSC-like cells in patients who responded to HMA/VEN treatment compared to non-responders (FIG. 4B). To assess response duration, the number of HMA/VEN cycles was used as a proxy for disease-free survival (DFS) as ongoing VEN treatment indicates ongoing response. Patients who moved on to receive allogenic stem cell transplantations in CR or stopped the treatment due to other reasons than disease progression were censored. Strikingly, a significantly longer HMA/VEN response in patients with above median response scores (>0.4) compared to patients with below median response scores (<0.4) (FIG. 4C) was observed. These findings were validated in a second cohort (n=19), where response scores in LSC-like cells were again significantly higher in patients who responded to HMA/VEN treatment (FIG. 4D). Moreover, the duration of HMA/VEN treatment in cohort 2 was again longer for patients with above median response scores, suggesting longer lasting sensitivity (FIG. 4E). When the two cohorts were combined, the response scores of responders remained significantly higher compared to non-responders (FIG. 4F). Furthermore, the prognostic accuracy of the response score in the combined cohorts with ROC analysis was assessed and a ROC value of 0.95 was observed. This indicates that the response score has high accuracy to predict HMA/VEN response (FIG. 4G). This was also observed in the probability of ongoing HMA/VEN treatment of the combined cohorts where patients with above median scores stayed on treatment longer (FIG. 4H). The observed separation between responders and non-responders was not observed when the response score was calculated on bulk, especially in monocytic AML samples (FIG. 8G-I). Of note, the response score showed no difference between patients who did or did not respond to standard induction therapy, highlighting its specificity to predict response to VEN based therapy (FIG. 8H). Next, it was assessed whether differences in the response scores within patients who showed initial response to HMA/VEN would also reveal distinction in regards to response duration. Indeed, responders with above median response scores (>0.4) stayed longer on HMA/VEN compared to responders with lower response scores (<0.4), showing that the response score can be used to distinguish patients with long-lasting response to HMA/VEN (FIG. 4I). Finally, multivariant analysis on all 37 studied patients, including response score as a variable was performed. In univariant analysis, response score and CK were the only significant parameters with trends in IDH1/2. Splicing factor and TET2 mutations. Multivariant linear regression was performed on these 5 parameters and it was found that only the response score was still a significant predictor with an Odds ratio of 10.2 per 0.1 points (FIG. 4J). Taken together, the response score is able to predict patient's initial response to HMA/VEN with an accuracy superior to mutational profiling and can select for patients with longer response durations. These attributes make the response score a readily and globally accessible, cheap tool with low turnover time to guiding therapeutic decision making in the move to bring HMA/VEN to first line therapy for selected fit patients.

### Example 12: Rapid and Robust Prediction of Clinical Response and Remission Duration with LSC-Like Response Score

Last, to gain insight also into the genetic profiles of AML patients with different response scores, clinical structural variant (SV) analysis and targeted mutational profiling was correlated with the response score in 73 diagnostic AML patient samples (FIG. 4K, FIG. 8I). It was observed that patients with low response score harbored a higher number of SVs compared to patients with high response scores (FIG. 4L). Moreover, mutations in JAK2 or CALR exclusively had low response score (FIG. 4M), which is in line with the association of prior MDS/MPN with clinical refractoriness to HMA/VEN, although sample size was small (FIG. 1C-D). On the other hand, patients with high response scores were enriched for IDH1/2 and splicing mutations (FIG. 4M) suggesting that that specific genetic alterations have a role in differential dependency on BCL-2 family proteins. Importantly not all patients with CK had low or all IDH1/2 mutant high response score, highlighting the value of patient individualized assessment. Taken together, the response score culminates the impact of the correlatively associated mutational background and other non-genetic, potentially elusive, factors as it provides the causal molecular reason determining HMA/VEN therapy response.

In the following Table 2, response scores calculated according to the method referred to above are compared to individual responses for 14 patients.

| | Response Prediction | | | | | |
|---|---|---|---|---|---|---|
| | (response score) | | | Response (% of LSC alive after 24 h relative to | | |
| AML | | | MCL-1- | untreated controls) | | |
| Patient ID | Venetoclax | Navitoclax | Venetoclax | Venetoclax/Aza | Navitoclax/Aza | Venetoclax/MCL-1 |
| D1 | 0.9 | 1.6 | 0.6 | 9.8 | 7.8 | 35.6 |
| D50 | 1.4 | 1.1 | 1.3 | 0.7 | 3.2 | 8.9 |
| D53 | 0.9 | 1.0 | 0.9 | 11.3 | 25.4 | 51.3 |
| D56 | 0.9 | 0.6 | 1.7 | 23.2 | 38.5 | 14.0 |
| D57 | 0.2 | 0.9 | 0.4 | 71.8 | 35.7 | 104.8 |
| D59 | 1.5 | 1.0 | 1.5 | 33.5 | 36.1 | 48.5 |
| D60 | 1.6 | 2.0 | 0.9 | 0.1 | 0.2 | 0.3 |
| D61 | 1.0 | 0.8 | 1.3 | 27.2 | 69.5 | 61.9 |
| D62 | 0.5 | 1.2 | 0.5 | 76.7 | 65.1 | 79.4 |
| D63 | 0.5 | 0.8 | 0.7 | 28.0 | 17.7 | 22.8 |
| D64 | 0.5 | 1.2 | 0.5 | 29.6 | 18.0 | 33.4 |
| D67 | 0.6 | 1.0 | 0.6 | 5.6 | 20.9 | 41.8 |
| D71 | 0.2 | 0.7 | 0.5 | 29.9 | 7.4 | 60.6 |
| D72 | 0.9 | 1.6 | 0.6 | 25.8 | 5.8 | 18.4 |
| | | | Best Treatment | | | |
| | | | Prediction | 71.42857143 | | |
| | | | Correct % | | | |
| | | | Prediction | 28.57142857 | | |
| | | | Wrong % | | | |
| | | | By Chance % | 33.3 | | |

response score number and % response in bold indicates correct predictions
response score number and % response in italic indicates incorrect predictions

$$Venetoclax response score : BCL - 2 / ( MCL - 1 + BCL - xL )$$

$$Navitoclax response score : 0.5 ( BCL - 2 + BCL - xL ) / MCL - 1$$

$$MCL - 1 - Venetoclax response score : 0.5 ( MCL - 1 + BCL - 2 ) / BCL - xL$$

The amounts used for calculating the response scores are typically normalized amounts as described elsewhere herein.

In the above Table 2, the Response scores for Venetoclax, Navitoclax and MCL-1-Venetoclax are all three calculated. It is apparent that by comparing the said response scores, the prediction can be even improved compared to the calculation of a single score. As will be apparent from Table 2, above, the highest response score among the three scores (indicated in bold) is also for a beneficial BCL family inhibitor therapy, i.e. if the Venetoclax response score is highest among the three scores, Venetoclax is beneficial, if the Navitoclax response score is highest among the three scores, Navitoclax is beneficial etc. as confirmed by the percentage of LSC that are alive after 24 h relative to untreated controls.

### Example 13: Response Score in LSC-Like Cell Predict Clinical Response and Remission Duration of 5-AZA/VEN

While BCL-2 conveys sensitivity to VEN, MCL-1 and BCL-xL can promote survival independent of BCL-2. Thus, to additionally account for these factors contributing to resistance, all three proteins were incorporated into a singular response score, and termed it “Response Score”, which can be calculated for subpopulations defined by flow cytometry. Response Score calculates the ratio between the normalized MFI of the drug target (BCL-2) and the normalized MFI of the resistance factors (sum of MCL-1 and BCL-xL) as follows:

$$BCL - {2}^{Norm . MFI} / ( MCL - {1}^{Norm . MFI} + BCL - {xL}^{Norm . MFI} )$$

$$( for details see Example 1 , General Methods , supra ) .$$

To ensure consistent and comparable MFI measurements of samples processed and analyzed on separate days a reference AML sample was processed and run along with each cohort. Detector voltages were adjusted to keep MFI for each BCL-2, MCL-1 and BCL-xL of the LSC-like population in the reference sample constant. Small fluctuations of reference sample MFIs were adjusted by normalizing the measurement day's reference sample to match previous reference sample measurements. For each sample, normalized MFI for each BCL-2, MCL-1 and BCL-xL of the LSC-like population were divided by the respective median MFI of AML patients classified as responders within the cohort to obtain relative MFI-values (rel. MFI).

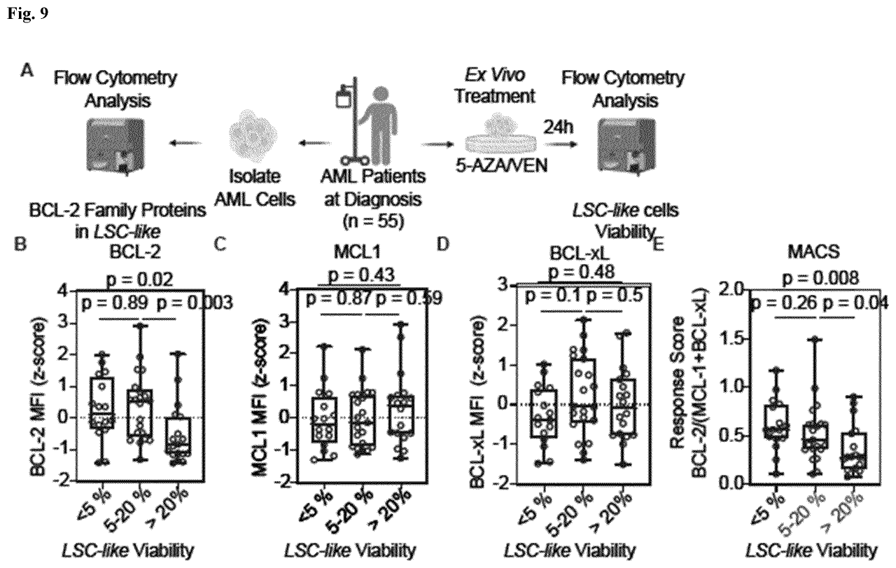

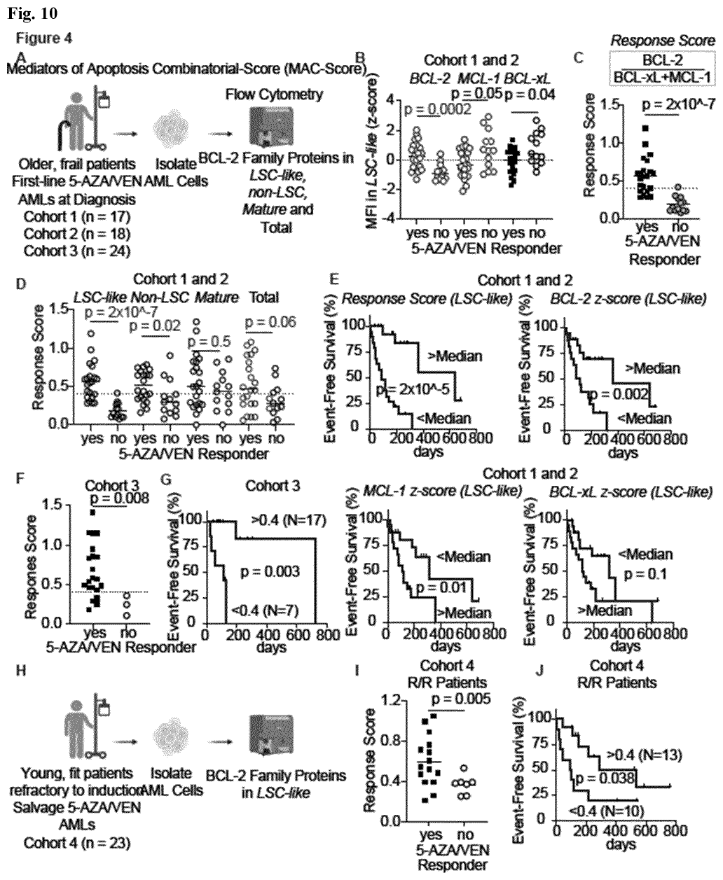

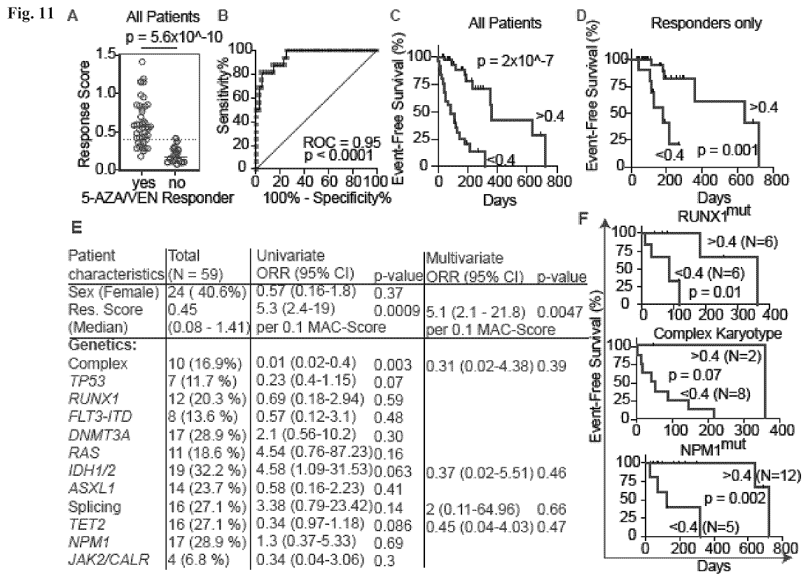

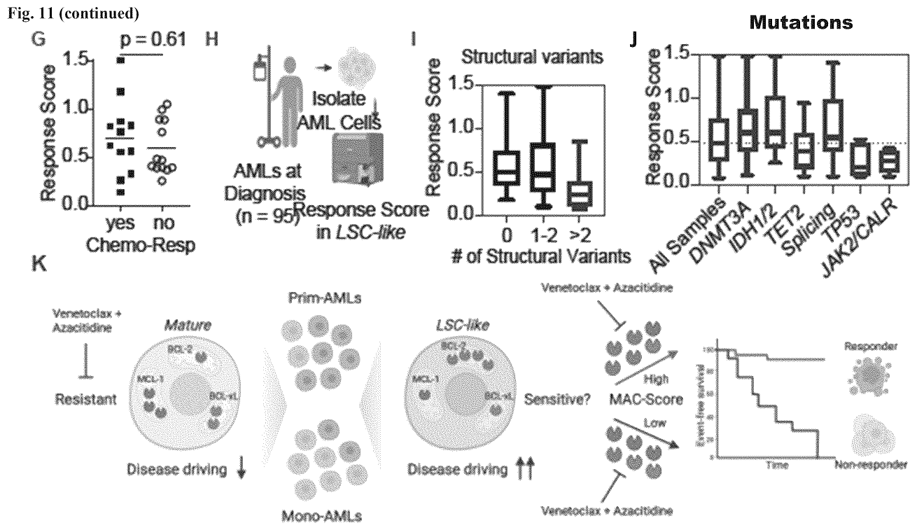

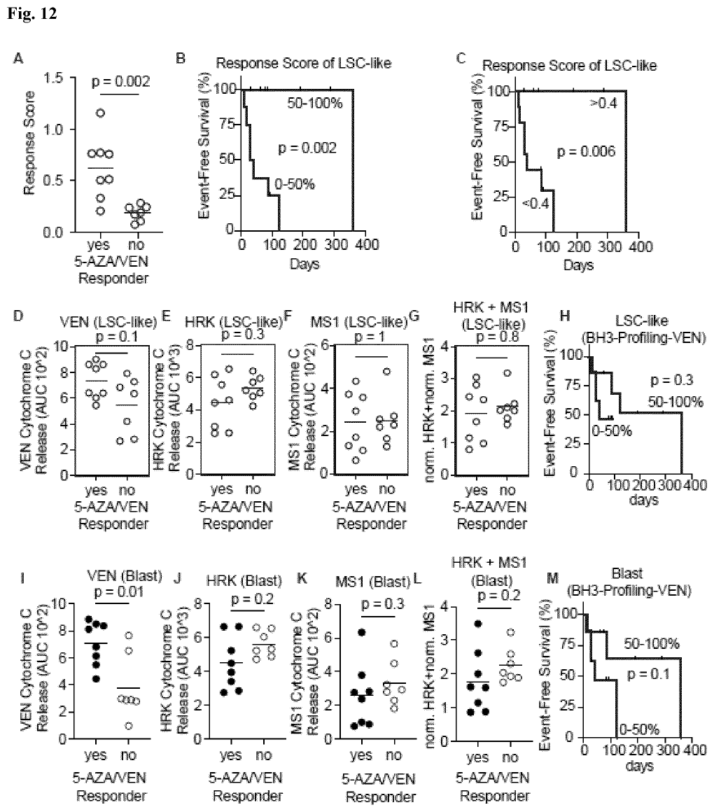

Accounting for the alternative inhibitors of apoptosis and potential mediators of VEN resistance, Response Score further improved separation between ex vivo 5-AZA/VEN sensitivity AML samples compared to individual protein levels (FIG. 9B-E). These data highlight the benefit of combinatorial assessment of BCL-2 family proteins specifically within LSC-like cells.

Next, it was assessed whether individual BCL-2 family protein levels or Response Score predict clinical response to 5-AZA/VEN treatment. Expression of BCL-2 family proteins BCL-2, MCL-1 and BCL-xL was analyzed together with cell surface expression profiling in 35 diagnostic samples from two independently processed multicenter cohorts (FIG. 10A, cohorts 1+2). Here, BCL-2 was significantly higher expressed in LSC-like cells of patients achieving CR, CRi or MLFS (responder), while MCL-1 and BCL-xL were higher expressed in patients with SD. PR or PD (non-responder) (FIG. 10B). Similar trends were observed in non-LSC cells and total blasts, while BCL-2 family expression in Mature cells did not differ between responders and non-responders. However, expression of neither BCL-2-family protein alone in LSC-like cells or in other subpopulations provided a clear separation between responders and non-responders, and the levels of all three proteins showed high intra-patient variability (FIG. 10B).

As Response Score outperformed the individual BCL-2 family proteins in predicting 5-AZA/VEN response ex vivo, Response Scores was calculated in LSC-like, non-LSC, Mature and total blast cells also in these two patient cohorts (FIG. 10C-D). Both cohorts individually and together showed significantly higher Response Scores in LSC-like cells derived from patients who responded to 5-AZA/VEN treatment compared to the LSC-like cells analyzed from non-responder patients (FIG. 10C-D). This phenomenon was also detected in total blasts but to a lower extent and without a clear separation between responders and non-responders (FIG. 10D). These findings indicate that Response Score in LSC-like cells outperforms expression of individual BCL-2 family proteins as a binary predictor of clinical response to 5-AZA/VEN.

To study response duration, event-free survival (EFS) of these two cohorts was assessed. Patients who discontinued treatment due to other reasons than disease progression were censored. For both cohorts a significantly longer 5-AZA/VEN response was observed in patients with median Response Scores >0.4 compared to patients with median Response Scores <0.4 in LSC-like cells (FIG. 10E). BCL-2 family proteins alone did not reach the same predictive value compared to Response score, even though high BCL-2 levels alone predicted longer and high MCL-1 shorter EFS if analyzed in LSC-like cells (FIG. 10E). Moreover, neither Response Score nor individual protein levels of the three BCL-2 members determined from the other subpopulations provided reasonable predictive power and were all outperformed by Response Score in LSC-like cells. These findings were validated in a third, independently processed cohort (n=24), in which Response Scores in LSC-like cells were again significantly higher in patients who responded to 5-AZA/VEN treatment and were low in patients that did not (FIG. 10F). EFS was also longer for patients with above median Response Scores, suggesting improved therapy response (FIG. 10G). Collectively, these findings support the concept that Response Score in LSC-like cells can accurately predict response to first-line treatment with 5-AZA/VEN in older, frail patients, outperforming prediction based on individual BCL-2 family proteins alone.

5-AZA/VEN is currently under investigation for first-line treatment of younger patients, and has shown efficacy as a salvage therapy option for relapsed and refractory patients. Therefore, it was tested if response to 5-AZA/VEN can be predicted in young, relapsed refractory patients, by assessing Response Scores in 23 AML samples receiving 5-AZA/VEN as salvage therapy (FIG. 10H). The samples were either biobanked at diagnosis or before the start of 5-AZA/VEN treatment. In this salvage setting, Response Scores above median were again highly predictive of binary clinical response and longer EFS, opening the path for the prospective assessment of biomarker-based choice of induction regimen (FIG. 10I-J). Together, Response Score enabled a robust identification of patients who benefit from 5-AZA/VEN as first-line and salvage therapy and is a step towards selecting the best therapy for each patient on an individual basis.

### Example 14: Response Score in LSC-Like Cells Outperforms BH3-Profiling

Measurement of apoptotic dependence by BH3-profiling has previously been proposed as a predictor of response to 5-AZA/VEN. To compare Response Score with BH3-profiling, 15 samples (7 non-responders vs. 8 responders) from the three cohorts above treated first-line with 5-AZA/VEN were selected and assessed both readouts. As expected, Response Score was highly predictive of binary clinical response (FIG. 12A). Moreover, EFS showed robust differences when stratified either by the median Response Score of the selected 15 samples or the previously established cutoff of 0.4 (FIG. 12B-C). In contrast, BH3-profiling in LSC-like cells based on VEN-, HRK- or MS1-induced cytochrome C release did not predict clinical response (FIG. 12D-F). Assessing the sum of the HRK- and MS1-induced cytochrome C release, as previously reported, did not improve the prediction (FIG. 12G). BH3-profiling in LSC-like cells also did not correlate with EFS in these patients (FIG. 12H). When total blasts were assessed, only VEN-induced cytochrome C release predicted binary clinical response but showed only a trend towards longer EFS (FIG. 12I-M). Importantly, the observed separation was weaker compared to Response Score in this patient cohort. In summary, Response Score is a robust alternative to BH3-profiling for VEN response prediction.

### Example 15: Combined Analysis of Response Scores in LSC-Like Cells Identifies Drivers of VEN Resistance

To further assess the prognostic accuracy of Response Score, the Response Scores in LSC-like cells was combined from all three first-line treated cohorts (cohorts 1, 2 and 3), extending the analysis to 59 patients treated first-line with 5-AZA/VEN. As expected, Response Scores were significantly higher in responders compared to non-responders (FIG. 11A). The prognostic accuracy of Response Score in the combined cohorts was assessed with ROC analysis and observed a ROC value of 0.95, revealing high accuracy to predict 5-AZA/VEN treatment response in patients (FIG. 11B). The combined EFS analysis of all 59 5-AZA/VEN first-line treated patients, showed a 4-fold prolongation of EFS in patients with above median scores with an EFS of 3 vs. 12 months (FIG. 11C). As some patients with Response Scores <0.4 responded to 5-AZA/VEN, it was next assessed whether Response Scores within responders would discriminate with regards to response duration. Indeed, responders with >0.4 Response Scores had a longer EFS with 5-AZA/VEN treatment compared to responders with Response Scores <0.4 with a median EFS of 6 vs. 12 months (FIG. 11D). This shows that Response Score can be used to distinguish patients with long-lasting response to 5-AZA/VEN.

Finally, to identify predictors of response to 5-AZA/VEN, logistic regression analysis was performed on all 59 first-line treated patients and included Response Score as one of the assessed variables. Using univariate analyses, Response Score and complex karyotype were identified as statistically significant parameters in this set of samples (FIG. 11E). Subsequently, multivariate analysis on all parameters with p<0.15 was performed and observed that Response Score remained as the sole predictor of response with an Odds ratio of 5.1 per 0.1 points (FIG. 11E). Response Score predicted EFS even within genetic subgroups like complex karyotype. RUNX1 or NPM1 mutated patients, highlighting its potential role in patient stratification beyond genetics (FIG. 11F). Of note, Response Score showed no difference between patients who did or did not respond to standard induction therapy, highlighting its specificity to predict response to Venetoclax-based therapies (FIG. 11G).

Taken together, Response Score predicts patient's initial response to 5-AZA/VEN with an accuracy superior to mutational profiling and can identify patients with long response durations.

### Example 16: Response Score Deconvolutes Response Heterogeneity within Mutational Patterns

To gain insights into the genetic profiles of AML patients with different Response Scores, clinical structural variant (SV) analysis and targeted mutational profiling with Response Scores for 95 diagnostic AML patient samples independent of the received treatment (FIG. 11H) were correlated. It was observed that patients with low Response Scores harbored a higher number of SVs compared to patients with high Response Scores (FIG. 11I). Moreover, AML patients with mutations in TET2 or JAK2 and CALR had exclusively low Response Scores (FIG. 11J). This is in line with the observed association of prior MDS/MPN diagnosis with clinical refractoriness to 5-AZA/VEN, although the sample size remains small (FIG. 1C). In contrast, patients with high Response Scores were enriched for IDH1/2, DNMT3A and splicing mutations (FIG. 11J), suggesting that specific genetic alterations affect the differential dependency of LSC-like cells on BCL-2 family proteins. Importantly, the heterogeneity of Response Scores within complex karyotypic or IDH1/2-mutant AMLs and the associated 5-AZA/VEN response, highlights the value of patient individualized assessment. Taken together, Response-Scoring in LSC-like cells aggregates the impact of the correlatively associated mutational background and other still elusive non-genetic factors by providing causality-based information that predicts patient response to 5-AZA/VEN therapy (FIG. 11K).

### CITED LITERATURE

Konopleva, M., Pollyea, D. A., Potluri, J., Chyla, B., Hogdal, L., Busman, T., McKeegan, E., Salem, A. H., Zhu, M., Ricker, J. L., et al. (2016). Efficacy and Biological Correlates of Response in a Phase II Study of Venetoclax Monotherapy in Patients with Acute Myelogenous Leukemia. Cancer Discov 6, 1106-1117
DiNardo, C. D., Jonas, B. A., Pullarkat, V., Thirman, M. J., Garcia. J. S., Wei, A. H., Konopleva, M., Dohner, H., Letai, A., Fenaux, P., et al. (2020a). Azacitidine and Venetoclax in Previously Untreated Acute Myeloid Leukemia. N Engl J Med 383, 617-629.
DiNardo, C. D., Lachowiez, C. A., Takahashi, K., Loghavi, S., Xiao, L., Kadia, T., Daver, N., Adeoti, M., Short, N. J., Sasaki, K., et al. (2021). Venetoclax Combined With FLAG-IDA Induction and Consolidation in Newly Diagnosed and Relapsed or Refractory Acute Myeloid Leukemia J Clin Oncol 39, 2768-2778.;
Garcia, J. S., Kim, H. T., Murdock, H. M., Cutler, C. S., Brock, J., Gooptu, M., Ho, V. T., Koreth. J., Nikiforow, S., Romee, R., et al. (2021). Adding Venetoclax to fludarabine/busulfan RIC transplant for high-risk MDS and AML is feasible, safe, and active. Blood Adv 5, 5536-5545
Cherry, E. M., Abbott, D., Amaya, M., McMahon, C., Schwartz, M., Rosser. J., Sato, A., Schowinsky, J., Inguva, A., Minhajuddin, M., et al. (2021). Venetoclax and azacitidine compared with induction chemotherapy for newly diagnosed patients with acute myeloid leukemia. Blood Adv 5, 5565-5573.
Dohner, H., Estey, E., Grimwade, D., Amadori, S., Appelbaum, F. R., Buchner, T., Dombret, H., Ebert, B. L., Fenaux, P., Larson, R. A., et al. (2017). Diagnosis and management of AML in adults: 2017 ELN recommendations from an international expert panel. Blood 129, 424-447.
Cai, S. F., Chu, S. H., Goldberg, A. D., Parvin, S., Koche, R. P., Glass, J. L., Stein, E. M., Tallman, M. S., Sen, F., Famulare, C. A., et al. (2020). Leukemia Cell of Origin Influences Apoptotic Priming and Sensitivity to LSD1 Inhibition. Cancer Discov 10, 1500-1513.
Bhatt, S., Pioso, M. S., Olesinski, E. A., Yilma, B., Ryan, J. A., Mashaka, T., Leutz, B., Adamia, S., Zhu, H., Kuang, Y., et al. (2020). Reduced Mitochondrial Apoptotic Priming Drives Resistance to BH3 Mimetics in Acute Myeloid Leukemia. Cancer Cell 38, 872-890 e876.
Kuusanmaki, H., Leppa, A. M., Polonen, P., Kontro, M., Dufva, O., Deb, D., Yadav, B., Bruck, O., Kumar, A., Everaus, H., et al. (2020). Phenotype-based drug screening reveals association between Venetoclax response and differentiation stage in acute myeloid leukemia. Haematologica 105, 708-720.
Pei, S., Pollyea, D. A., Gustafson. A., Stevens, B. M., Minhajuddin, M., Fu, R., Riemondy, K. A., Gillen, A. E., Sheridan, R. M., Kim, J., et al. (2020). Monocvtic Subclones Confer Resistance to Venetoclax-Based Therapy in Patients with Acute Myeloid Leukemia. Cancer Discov 10, 536-551.
DiNardo, C. D., Maiti, A., Rausch, C. R., Pemmaraju, N., Naqvi, K., Daver, N. G., Kadia, T. M., Borthakur, G., Ohanian, M., Alvarado, Y., et al. (2020b). 10-day decitabine with Venetoclax for newly diagnosed intensive chemotherapy ineligible, and relapsed or refractory acute myeloid leukaemia: a single-centre, phase 2 trial. Lancet Haematol 7, e724-e736.
Stahl, M., Menghrajani, K., Derkach, A., Chan. A., Xiao, W., Glass, J., King, A. C., Daniyan, A. F., Famulare, C., Cuello, B. M., et al. (2021). Clinical and molecular predictors of response and survival following Venetoclax therapy in relapsed/refractory AML. Blood Adv 5, 1552-1564.

1.-57. (canceled)

58. A method for assessing response to a BCL-family inhibitor therapy in a subject suffering from cancer, preferably a hematopoietic cancer, comprising the steps of:
(a) determining the amounts of biomarkers BCL-2, BCL-xL, and MCL-1 in a tumor driving cell population, preferably leukemic stem cell (LSC) population, in a sample of the subject; and
(b) comparing the amounts of the biomarkers to a reference, whereby the response to a BCL-family inhibitor therapy is assessed.

59. The method of claim 58, wherein the assessing response to a BCL-family inhibitor therapy comprises identifying whether a subject will benefit from the treatment by BCL-2 inhibitor.

60. The method of claim 59, wherein comparing the biomarkers to a reference comprises calculating the ratio of the amount of BCL-2 to the combined amounts of BCL-xL and MCL-1 to obtain a prediction score, and comparing the prediction score to a reference, preferably wherein calculating the prediction score is based on using the following formula:
$$prediction score = BCL - 2 / ( MCL - 1 + BCL - xL ) .$$

61. The method of claim 60, wherein a prediction score larger than the reference is indicative of a subject that will benefit from the treatment with the BCL-2 inhibitor, or wherein a predictive score lower than the reference is indicative of a subject that will not benefit from the treatment with the BCL-2 inhibitor.

62. The method of claim 58, wherein the reference is a reference value derived from a non-responder population, preferably, wherein the reference is between about 0.6 and about 1.0, preferably is about 0.8.

63. The method of claim 58, wherein the assessing response to a BCL-family inhibitor therapy comprises identifying whether a subject will benefit from the treatment with a BCL-xL and/or MCL-1 inhibitor.

64. The method of claim 63, wherein comparing the biomarkers to a reference comprises calculating the ratio of the half of the combined amounts of BCL-2 and BCL-xL to the amount of MCL-1 to obtain a prediction score and comparing the prediction score to a reference, preferably wherein calculating the prediction score is based on using the following formula:
$$prediction score = 0.5 ( BCL - 2 + BCL - xL ) / MCL - 1.$$

65. The method of claim 64, wherein a prediction score larger than the reference is indicative for a subject that will benefit from the treatment by a BCL-xL and/or MCL-1 inhibitor, or wherein a predictive score lower than the reference is indicative for a subject that will not benefit from the treatment by a BCL-xL and/or MCL-1 inhibitor.

66. The method of claim 64, wherein the reference is a reference value derived from a non-responder population, preferably, wherein the reference is between about 0.6 and about 1.0, preferably is about 0.8.

67. The method of claim 58, wherein the assessing response to a BCL-family inhibitor therapy comprises identifying whether a subject will benefit from the treatment by BCL-2 inhibitor and at least one MCL-1 inhibitor.

68. The method of claim 67, wherein comparing the biomarkers to a reference comprises calculating the ratio of the amount of BCL-2 to the combined amounts of BCL-xL and MCL-1 to obtain a prediction score and comparing the prediction score to a reference, preferably, wherein calculating the prediction score is based on using the following formula:
$$prediction score = 0.5 ( MCL - 1 + BCL - 2 ) / BCL - xL .$$

69. The method of claim 68, wherein a prediction score larger than the reference is indicative for a subject that will benefit from the treatment by a BCL-2 inhibitor and at least one MCL-1 inhibitor, or wherein a prediction score lower than the reference is indicative for a subject that will not benefit from the treatment by a BCL-2 inhibitor and at least one MCL-1 inhibitor.

70. The method of claim 68 wherein the reference is a reference value derived from non-responder population, preferably, wherein the reference is between about 0.6 and about 1.0, preferably is about 0.8.

71. The method of claim 58, wherein the LSC population is characterized by increased expression of at least one biomarker selected from the group consisting of GPR56, CD34 and BCL-2, preferably, wherein the expression is increased compared to the expression of the at least one biomarker in monocyte-like AML cells.

72. A method for treating a subject suffering from cancer, preferably a hematopoietic cancer, with a BCL-2 inhibitor, the method comprising assessing a response to the BCL-2 inhibitor for the subject by the method of claim 59, and administering the BCL-2 inhibitor to the subject if the subject is assessed to benefit from therapy using the BCL-2 inhibitor.

73. A method for treating a subject suffering from cancer, preferably a hematopoietic cancer, with a BCL-xL and/or MCL-1 inhibitor, the method comprising assessing a response to the BCL-xL and/or MCL-1 inhibitor for the subject by the method of claim 63, and administering the BCL-xL and/or MCL-1 inhibitor to the subject if the subject is assessed to benefit from therapy using the BCL-xL and/or MCL-1 inhibitor.

74. A method for treating a subject suffering from cancer, preferably a hematopoietic cancer, with a BCL-2 inhibitor in combination with at least one MCL-1 inhibitor, the method comprising:
(a) assessing a response to the BCL-2 inhibitor in combination with the at least one MCL-1 inhibitor for the subject by the method of claim 67, and
(b) administering the BCL-2 inhibitor in combination with the at least one MCL-1 inhibitor to the subject if the subject is assessed to benefit from therapy using the BCL-2 inhibitor in combination with the at least one MCL-1 inhibitor.

75. A device for assessing response to a BCL-family inhibitor therapy in a subject suffering from cancer, preferably a hematopoietic cancer, comprising:
(a) an analyzing unit capable of determining the amounts of the biomarkers BCL-2, BCL-xL, and MCL-1 in a tumor driving cell population, preferably leukemic stem cell (LSC) population, in a sample of the subject; and
(b) an evaluation unit comprising a data processor capable of comparing the amounts of the biomarkers to a reference, whereby the response to a BCL-family inhibitor therapy is assessed.

76. A kit for assessing response to a BCL-family inhibitor therapy in a subject suffering from cancer, preferably a hematopoietic cancer, comprising detection molecules for determining the amounts of the biomarkers BCL-2, BCL-xL, and MCL-1 in a tumor driving cell population, preferably leukemic stem cell (LSC) population, in a sample of the subject.
